(19) United States
(12) Patent Application Publication
(10) Pub. No.: US 2026/0297544 A1
(43) Pub. Date: Oct. 1, 2026

(54) MODIFIED HELICASES

(71) Applicant: Oxford Nanopore Technologies PLC, Oxford (GB)

(72) Inventors: Matthew Robertson Herbert, Oxford (GB); Elizabeth Ann Snell, Oxford (GB); Richard Charles Foster, Oxford (GB); Rebecca Victoria Boven, Oxford (GB); Mark John Bruce, Oxford (GB); Joseph Hargreaves Lloyd, Oxford (GB); Jaskrian Singh Sabharwal, Oxford (GB)

(73) Assignee: Oxford Nanopore Technologies PLC, Oxford (GB)

(21) Appl. No.: 19/482,936

(22) PCT Filed: May 13, 2024
(86) PCT No.: PCT/EP2024/063118

§ 371 Date: Nov. 10, 2025

(30) Foreign Application Priority Data
May 12, 2023 (GB) 2307141.8

(51) Int. Cl. C12N 9/14; G01N 33/53

(52) U.S. Cl. CPC C12N 9/14; G01N 33/5308; C07K 2319/80; C12Y 306/04013; G01N 2333/914

(57) ABSTRACT

The invention relates to modified helicases which are capable of controlling the movement of polynucleotides with increased speed. The modified helicases are particularly useful for sequencing polynucleotides.

### RELATED APPLICATIONS

This application is a national stage filing under 35 U.S.C. § 371 of international application number PCT/EP2024/063118, filed May 13, 2024, which claims the benefit of United Kingdom application number GB 2307141.8, filed May 12, 2023, each of which is herein incorporated by reference in its entirety.

### TECHNICAL FIELD

The invention relates to modified helicases which are capable of controlling the movement of polynucleotides with increased speed. The modified helicases are particularly useful for sequencing polynucleotides.

### BACKGROUND

Pore sensing is an approach to analyte detection and characterisation that relies on the observation of individual binding or interaction events between the analyte molecules and an ion conducting channel. pore sensors can be created by placing a single pore of nanometre dimensions in an electrically insulating membrane and measuring voltage-driven ion currents through the pore in the presence of analyte molecules. The presence of an analyte inside or near the pore will alter the ionic flow through the pore, resulting in altered ionic or electric currents being measured over the channel. The identity of an analyte is revealed through its distinctive current signature, notably the duration and extent of current blockades and the variance of current levels during its interaction time with the pore. Analytes can be organic and inorganic small molecules as well as various biological or synthetic macromolecules and polymers including polynucleotides, polypeptides, and polysaccharides. pore sensing can reveal the identity and perform single molecule counting of the sensed analytes but can also provide information on the analyte composition such as nucleotide, amino acid, or glycan sequence, as well as the presence of base, amino acid, or glycan modifications such as methylation and acylation, phosphorylation, hydroxylation, oxidation, reduction, glycosylation, decarboxylation, deamination and more. pore sensing has the potential to allow rapid and cheap polynucleotide sequencing, providing single molecule sequence reads of polynucleotides of tens to millions bases length.

Two of the essential components of polymer characterization using pore sensing are (1) the control of polymer movement through the pore and (2) the discrimination of the composing building blocks as the polymer is moved through the pore. The movement of the polymer through the pore is typically controlled using an enzyme, such as a helicase. Suitable enzymes are disclosed in WO 2013/057495, WO 2013/098562, WO2013098561, WO 2014/013260, WO 2014/013259, WO 2014/013262, and WO 2015/055981. All of these are incorporated by reference in their entirety. WO 2014/013260 discloses modified helicases in which two or more parts of the helicase are connected via one or more attached linkers to form a covalently closed structure around the polynucleotide binding domain. This reduces the ability of the modified helicase to unbind from the polynucleotide and therefore increases its processivity (i.e., its ability to control the movement of long polynucleotides). There is a need for new enzymes and helicases for use in analyte characterisation.

### SUMMARY OF THE INVENTION

The invention relates to covalently closed NS3 helicases with longer attachment linkers. The invention also relates to methods of increasing the speed at which a modified helicase is capable of controlling the movement of the polynucleotide and improved helicases produced using these methods. The invention also relates to using the improved helicases of the invention for analyte characterisation.

The inventors have surprisingly shown that increasing the length of one or more attachment linkers that form a covalently closed structure around the polynucleotide binding domain of a modified helicase increases the speed at which the modified helicase can control the movement of a polynucleotide.

The invention therefore provides a modified NS3 helicase comprising a polynucleotide binding domain, wherein two or more parts of the helicase are connected via one or more attached linkers to form a covalently closed structure around the polynucleotide binding domain, wherein the one or more attached linkers are greater than about 8.0 angstroms (Å) in length and wherein the modified helicase retains its ability to control the movement of the polynucleotide.

The invention also provides:
a modified NS3 helicase comprising a polynucleotide binding domain, wherein two or more parts of the helicase are connected via one or more attached linkers to form a covalently closed structure around the polynucleotide binding domain, wherein the NS3 helicase is derived from a dengue virus (NS3-DV), a yellow fever virus (NS3-YFV), or a hepatitis C virus (NS3-HCV), and wherein the modified helicase retains its ability to control the movement of the polynucleotide,
a method of increasing the speed at which a helicase is capable of controlling the movement of the polynucleotide, wherein the helicase comprises a polynucleotide binding domain, the method comprising modifying the helicase by connecting two or more parts of the helicase via one or more attached linkers greater than about 8.0 angstroms (Å) in length to form a covalently closed structure around the polynucleotide binding domain,
a modified helicase which is capable of controlling the movement of a polynucleotide with an increased speed produced using a method of the invention,
a construct comprising a modified helicase of the invention and an additional polynucleotide binding moiety, wherein the modified helicase is attached to the polynucleotide binding moiety and the construct has the ability to control the movement of a polynucleotide,
use of a modified helicase of the invention or a construct of the invention for controlling the movement of an analyte,
a method of controlling the movement of an analyte, comprising contacting the analyte with a modified helicase of the invention or a construct a of the invention and thereby controlling the movement of the analyte,
a method of determining the presence, absence or one or more characteristics of a target analyte, comprising: (a) contacting the target analyte with a pore and a modified helicase of the invention or a construct of the invention such that the modified helicase or construct controls the movement of the target analyte through the pore; and (b) taking one or more measurements as the target analyte moves with respect to the pore wherein the measurements are indicative of one or more characteristics of the target analyte and thereby determining the presence, absence or one or more characteristics of the target analyte,
a method of forming a sensor for characterising a target analyte, comprising forming a complex between (a) a pore and (b) a modified helicase of the invention or a construct of the invention and thereby forming a sensor for characterising the target analyte,
a sensor for characterising a target analyte, comprising a complex between (a) a pore and (b) a modified helicase of the invention or a construct of the invention,
a kit for characterising a target analyte comprising (a) a modified helicase of the invention or a construct of the invention and (b) one or more loading moieties,
an apparatus for characterising target analytes in a sample, comprising (a) a plurality of pores and (b) a plurality of modified helicases of the invention or a plurality of constructs of the invention,
a method of producing a modified helicase of the invention, comprising (a) providing a helicase and (b) modifying the helicase with one or more attached linkers to produce a modified helicase of the invention,
a series of two or more helicases attached to a polynucleotide, wherein at least one of the two or more helicases is a modified helicase of the invention,
a system comprising (a) a membrane comprising a pore, (c) a modified helicase of the invention or a construct according of the invention, (b) means for applying a potential across the membrane and (c) means for detecting electrical or optical signals across the membrane.

### DESCRIPTION OF THE FIGURES

FIG. 1: Translocation duration (seconds) of variant 1 with various different linkers.

FIG. 2: Speed (in bases per second) of variant 1 with various different linkers.

FIG. 3: Translocation duration (seconds) of variants 1, 2 and 3 with different linkers.

FIG. 4: Speed (in bases per second) of variants 1, 2 and 3 with different linkers.

### DETAILED DESCRIPTION

All publications, patents and patent applications cited herein, whether supra or infra, are hereby incorporated by reference in their entirety. All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the invention contained in the specification, the specification is intended to supersede and/or take precedence over any such contradictory material.

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein.

In addition, as used in this specification and the appended claims, the singular forms “a”, “an”, and “the” include plural referents unless the content clearly dictates otherwise. Thus, for example, reference to “a polynucleotide” includes two or more polynucleotides, reference to “a polynucleotide binding protein” includes two or more such proteins, reference to “a modified helicase” includes two or more modified helicases, reference to “a monomer” refers to two or more monomers, reference to “a pore” includes two or more pores and the like.

In all of the discussion herein, the standard one letter codes for amino acids are used. These are as follows: alanine (A), arginine (R), asparagine (N), aspartic acid (D), cysteine (C), glutamic acid (E), glutamine (Q), glycine (G), histidine (H), isoleucine (I), leucine (L), lysine (K), methionine (M), phenylalanine (F), proline (P), serine(S), threonine (T), tryptophan (W), tyrosine (Y) and valine (V). Standard substitution notation is also used, i.e., I199C means that I at position 199 is replaced with C.

Unless specifically defined herein, all terms used herein have the same meaning as they would to one skilled in the art of the present invention. Practitioners are particularly directed to Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016), for definitions and terms of the art. The definitions provided herein should not be construed to have a scope less than understood by a person of ordinary skill in the art.

“About” as used herein when referring to a measurable value such as an amount and the like, is meant to encompass variations of ±20% or ±10%, more preferably ±5%, even more preferably ±1%, and still more preferably ±0.1% from the specified value, as such variations are appropriate to perform the disclosed methods. Any statement herein including the term “about” includes the same feature without the term. For instance, the one or more linkers being greater than about 8.0 angstroms (Å) in length includes the one or more linkers being greater than 8.0 angstroms (Å) in length.

In the paragraphs herein where different positions are separated by the/symbol, the/symbol means “and” such that I199C/M439C is I199C and M439C.

The general definitions in WO 2019/002893 and WO 2014/013260 are incorporated by reference herein in their entirety.

### Modified NS3 Helicases

The invention provides modified NS3 helicases. The modified NS3 helicases are useful for controlling the movement of a target analyte, such as a polynucleotide. The modified NS3 helicase is based on an unmodified, monomeric NS3 helicase. The modified NS3 helicase is based on an unmodified helicase comprising a polynucleotide binding domain. The polynucleotide binding domain typically comprises in at least one conformational state an opening through which a polynucleotide can unbind from the helicase. In accordance with the invention, the NS3 helicase is modified to form a covalently closed structure around the polynucleotide binding domain. The covalently closed structure does not prevent the NS3 helicase from binding to a polynucleotide. For instance, the NS3 helicase may bind to a polynucleotide at one of its termini. The covalently closed structure decreases the ability of the polynucleotide to unbind or disengage from the NS3 helicase, particularly from internal nucleotides of the polynucleotide. This allows the modified NS3 helicase to remain bound to the polynucleotide for longer. The modified NS3 helicase has the ability to control the movement of a polynucleotide. The modified helicase is artificial or non-natural.

The ability of a helicase to bind to and unbind from a polynucleotide can be determined using any method known in the art. Suitable binding/unbinding assays include, but are not limited to, native polyacrylamide gel electrophoresis (PAGE), fluorescence anisotropy, calorimetry, and Surface plasmon resonance (SPR, such as Biacore™). The ability of a helicase to unbind from a polynucleotide can of course be determined by measuring the time for which the helicase can control the movement of a polynucleotide. This may also be determined using any method known in the art. The ability of a helicase to control the movement of a polynucleotide is typically assayed in a pore system, such as the ones described below. The ability of a helicase to control the movement of a polynucleotide can be determined as described in the Examples.

In a first embodiment, the modified NS3 helicase comprises a polynucleotide binding domain, wherein two or more parts of the helicase are connected via one or more attached linkers to form a covalently closed structure around the polynucleotide binding domain, wherein the one or more attached linkers are greater than about 8.0 angstroms (Å) in length and wherein the modified helicase retains its ability to control the movement of the polynucleotide. In all instances herein, one or more attached linkers includes one attached linker.

In this first embodiment, the modified helicase may also be defined in any of the following ways:
The invention provides a modified NS3 helicase comprising a polynucleotide binding domain which comprises in at least one conformational state an opening through which a polynucleotide can unbind from the helicase, wherein the helicase is modified with one or more attached linkers such that it is capable of forming a covalently closed structure around the polynucleotide, wherein the one or more attached linkers are greater than about 8.0 angstroms (Å) in length and wherein the helicase retains its ability to control the movement of the polynucleotide.
The invention also provides a complex comprising (i) a NS3 helicase that comprises a polynucleotide binding domain, and (ii) a target polynucleotide bound to the polynucleotide binding domain, wherein two amino acid residues that are located in different structural domains on the surface of the NS3 helicase surrounding the polynucleotide binding domain are artificially covalently connected via a linkage between the two amino acid residues, such that the helicase has a covalently closed structure, wherein the linkage is greater than about 8.0 angstroms (Å) in length, and wherein the bound target polynucleotide is encircled by the covalently-closed structure.
The invention also provides a modified NS3 helicase comprising a polynucleotide binding domain which comprises in at least one conformational state an opening through which a polynucleotide can unbind from the helicase, wherein the helicase is modified such that two or more parts of the helicase are covalently connected using one or more attached linkers and wherein the helicase retains its ability to control the movement of the polynucleotide, wherein the one or more attached linkers connect two amino acid residues that are located in different structural domains on the surface of the NS3 helicase surrounding the polynucleotide binding domain and wherein the one or more attached linkers are greater than about 8.0 angstroms (Å) in length. The at least one amino acid of the two amino acid residues is preferably substituted with cysteine, a non-natural amino acid or 4-azido-L-phenylalamine (Faz).

The one or more attached linkers are or the linkage is greater than about 8 angstroms (Å) in length. The one or more attached linkers are or the linkage is preferably greater than about 8.5 Å, greater than about 9.0 Å, greater than about 9.5 Å, greater than about 10.0 Å, greater than about 10.5 Å, greater than about 11.0 Å, greater than about 11.5 Å, greater than about 12.0 Å, greater than about 12.5 Å, greater than about 13.0 Å, greater than about 13.5 Å, greater than about 14.0 Å, greater than about 14.5 Å, greater than about 15.0 Å, greater than about 15.5 Å, greater than about 16.0 Å, or greater than about 16.5 Å, greater than about 17.0 Å, greater than about 17.5 Å, greater than about 18.0 Å, greater than about 18.5 Å, greater than about 19.0 Å, greater than about 19.5 Å, greater than about 20.0 Å, greater than about 20.5 Å, greater than about 20.9 Å, greater than about 21.0 Å, greater than about 21.5 Å, greater than about 22.0 Å, greater than about 22.5 Å, greater than about 23.0 Å, greater than about 23.5 Å, greater than about 24.0 Å, greater than about 24.5 Å, or greater than about 25.0 Å in length.

The one or more attached linkers are preferably at least about 10.0 Å in length. The one or more attached linkers are more preferably at least about 10.5 Å in length, at least about 11.0 Å in length, at least about 11.5 Å in length, at least about 12.0 Å in length, at least about 12.5 Å in length, at least about 13.0 Å in length, at least about 13.5 Å in length, at least about 14.0 Å in length, at least about 14.5 Å in length, at least about 15.0 Å in length, at least about 15.5 Å in length, at least about 16.0 Å in length, at least about 16.5 Å in length, at least about 17.0 Å in length, at least about 17.5 Å in length, at least about 18.0 Å in length, at least about 18.5 Å in length, at least about 19.0 Å in length, at least about 19.5 Å in length, at least about 20.0 Å in length, at least about 20.5 Å in length, at least about 20.9 Å in length, at least about 21.0 Å in length, at least about 21.5 Å in length, at least about 22.0 Å in length, at least about 22.5 Å in length, at least about 23.0 Å in length, at least about 23.5 Å in length, at least about 24.0 Å in length, at least about 24.5 Å in length, or at least about 25.0 Å in length.

The one or more attached linkers are or the linkage is preferably from about 8.5 Å to about 30.0 Å in length. The one or more attached linkers are or the linkage is preferably from about 9.0 Å to about 25.0 Å in length, from about 9.5 Å to about 22.0 Å in length, from about 9.75 Å to about 20.9 Å, from about 10.0 Å to about 20.0 Å in length, from about 10.5 Å to about 18.0 Å in length, from about 10.9 Å to about 18.0 Å in length, from about 10.5 Å to about 17.8 Å in length, or from about 10.9 Å to about 17.8 Å in length.

The one or more attached linkers are or the linkage is preferably about 10.9 Å, about 14.7 Å, about 17.8 Å, or about 20.9 Å in length. The one or more attached linkers are or the linkage is preferably about 10.9 Å, about 14.7 Å, or about 17.8 Å in length.

The length of the linker is typically measured based on the distance between carbon molecules in the spacer chain and their bond angles. Manufacturers of linkers typically provide an estimated length of the linkers. For instance, suitable linkers and their lengths are shown in Thermo Fisher Scientific's Crosslinking Technical Handbook: https://tools.thermofisher.com/content/sfs/brochures/1602163-Crosslinking-Reagents-Handbook.pdf

Suitable linkers for use in the invention are shown in the Table below. The one or more attached linkers or the linkage in the first embodiment may be selected from any of these linkers.

| Abbreviation | Linker Name | Spacer Arm (Å) | |
|---|---|---|---|
| BMB | 1,4-Bis-Maleimidobutane | 10.9 | Å |
| BMDB | 1,4-Bis-Maleimmidyl-2,3-dihydroxy-butane | 10.2 | Å |
| BMH | Bis-Maleimidohexane | 16.1 | Å |
| BMOE | Bis-Maleimidoethane | 8.0 | Å |
| BMPH | N-(β-Maleimidopropionic acid)hydrazide•TFA | 8.1 | Å |
| BM(PEG)₂ | 1,8-Bis-Maleimidodiethylene-glycol | 14.7 | Å |
| BM(PEG)₃ | 1,11-Bis-Maleimidotriethyleneglycol | 17.8 | Å |
| BS³ (Sulfo- | Bis (sulfosuccinimidyl)suberate | 11.4 | Å |
| DSS) | | | |
| BS³-d₀ | Bis (sulfosuccinimidyl)suberate-d0 | 11.4 | Å |
| BS³-d₄ | Bis (sulfosuccinimidyl)2,2,7,7-suberate-d4 | 11.4 | Å |
| BS(PEG)₅ | Bis (NHS)PEG₅ | 21.7 | Å |
| BS(PEG)₉ | Bis (NHS)PEG₉ | 35.8 | Å |
| BSOCOES | Bis (2-[succinimidoxycarbonyloxy]ethyl)sulfone | 13 | Å |
| DMA | Dimethyl adipimidate•2HCI | 8.6 | Å |
| DMP | Dimethyl pimelimidate•2HCI | 9.2 | Å |
| DMS | Dimethyl suberimidate•2HCl | 11 | Å |
| DSP | Dithiobis(succimidylpropionate) (Lomant's Reagent) | 12 | Å |
| DSS | Disuccinimidyl suberate | 11.4 | Å |
| DTBP | Dimethyl 3,3′-dithiobispropionimidate•2HCI | 11.9 | Å |
| DTME | Dithiobis-maleimidoethane | 13.3 | Å |
| DTSSP (Sulfo- | 3,3′-Dithiobis (sulfosuccinimidylpropionate) | 12 | Å |
| DSP) | | | |
| EGS | Ethylene glycol bis (succinimidylsuccinate) | 16.1 | Å |
| EMCA | N-ε-Maleimidocaproic acid | 9.4 | Å |
| EMCH | N-(ε-Maleimidocaproic acid)hydrazide | 11.8 | Å |
| EMCS | N-(ε-Maleimidocaproyloxy)succinimide ester | 9.4 | Å |
| KMUH | N-(κ-Maleimidoundecanoic acid)hydrazide | 19.0 | Å |
| LC-SDA | NHS-LC-Diazirine | 12.5 | Å |
| LC-SMCC | Succinimidyl 4-(N-maleimidomethyl) cyclohexane-1- | 16.2 | Å |
| | carboxy-(6-amidocaproate) | | |
| LC-SPDP | Succinimidyl 6-(3′-[2-pyridyl- | 15.7 | Å |
| | dithio]propionamido)hexanoate | | |
| MPBH | 4-(4-N-Maleimidophenyl)-butyric acid hydrazide•HCI | 17.9 | Å |
| Mts-Atf-Biotin | 2-[N2-(4-Azido-2,3,5,6-tetrafluorobenzoyl)-N 6-(6- | Mts-Atf 11.1 Å | |
| trifunctional | biotinamidocaproyl)-L-lysinyl]ethylmethanethiosulfate | Mts-Biotin 29.3 Å | |
| crosslinker | | Atf-Biotin 30.7 Å | |
| Mts-Atf-LC- | 2-{N2-[N6-(4-Azido-2,3,5,6-tetrafluorobenzoyl)-N 6- | Mts-Atf 21.8 Å | |
| Biotin | (6-biotinamidocaproyl)-L- | Mts-Biotin 29.3 Å | |
| trifunctional | lysinyl]}ethylmethanethiosulfate | Atf-Biotin 35.2 Å | |
| crosslinker | | | |
| NHS-PEG₄- | N-hydroxysuccinimide ester tetraoxapentadecane azide | 18.9 | Å |
| Azide | | | |
| NHS-PEG₁₂- | N-hydroxysuccinimide ester dodecaoxanonatriacontane | 47.3 | Å |
| Azide | azide | | |
| PDPH | 3-(2-Pyridyldithio)propionylhydrazide | 9.2 | Å |
| PEG₄-SPDP | 2-pyridyldithiol-tetraoxatetradecane-N- | 25.7 | Å |
| | hydroxysuccinimide | | |
| PEG₁₂-SPDP | 2-pyridyldithiol-tetraoxaoctatriacontane-N- | 54.1 | Å |
| | hydroxysuccinimide | | |
| PMPI | N-(p-Maleimidophenyl)isocyanate | 8.7 | Å |
| SDAD | NHS-SS-Diazirine | 13.5 | Å |
| SIAB | N-Succinimidyl(4-iodoacetyl)aminobenzoate | 10.6 | Å |
| SMCC | Succinimidyl 4-(N-maleimido-methyl)cyclohexane-1- | 8.3 | Å |
| | carboxylate | | |
| SM[PEG]₂ | NHS-PEG₂-Maliemide | 17.6 | Å |
| SM[PEG]₄ | NHS-PEG₄-Maliemide | 24.6 | Å |
| SM(PEG)₆ | NHS-PEG₆-Maleimide | 32.5 | Å |
| SM[PEG]₈ | NHS-PEG₈-Maliemide | 39.2 | Å |
| SM[PEG]₁₂ | NHS-PEG₁₂-Maliemide | 53.4 | Å |
| SM(PEG)₂₄ | NHS-PEG₂₄-Maleimide | 95.2 | Å |
| SMPB | Succinimidyl 4-(p-maleimido-phenyl)butyrate | 11.6 | Å |
| SMPH | Succinimidyl-6-(3-maleimidopropionamido)hexanoate | 14.2 | Å |
| SMPT | 4-Succinimidyloxycarbonyl-methyl-α-(2- | 20.0 | Å |
| | pyridyldithio)toluene | | |
| SPB | Succinimidyl-(4-psoralen-8-yloxy)butyrate | 8.5-9.5 | Å |
| Sulfo-EGS | Ethylene glycol bis (sulfo-succinimidyl succinate) | 16.1 | Å |
| Sulfo-EMCS | N-(ε-Maleimidocaproyloxy)sulfosuccinimide ester | 9.4 | Å |
| Sulfo-KMUS | N-(κ-Maleimidoundecanoyloxy)sulfosuccinimide ester | 16.3 | Å |
| Sulfo-LC-SDA | Sulfo-NHS-LC-Diazirine | 12.5 | Å |
| Sulfo-LC- | Sulfosuccinimidyl 6-(3′-[2-pyridyl- | 15.7 | Å |
| SPDP | dithio]propionamido)hexanoate | | |
| Sulfo- | Sulfosuccinimidyl 6-(4′-azido-2′- | 18.2 | Å |
| SANPAH | nitrophenylamino)hexanoate | | |
| Sulfo-SBED | Sulfo-NHS-(2-6-[Biotinamido]-2-(p-azidobezamido) | Sulfo-NHS ester | |
| trifunctional | | 13.7 Å Phenyl | |
| crosslinker | | azide 9.1 Å Biotin | |
| binds Avidin, | | 19.1 Å | |
| Strepavidin or | | | |
| NeutrAvidin | | | |
| Protein | | | |
| Sulf-SDAD | Sulfo-NHS-SS-Diazirine | 13.5 | Å |
| Sulfo-SIAB | Sulfosuccinimidyl(4-iodo-acetyl)aminobenzoate | 10.6 | Å |
| Sulfo-SMCC | Sulfosuccinimidyl 4-(N-maleimido- | 8.3 | Å |
| | methyl)cyclohexane-1-carboxylate | | |
| Sulfo-SMPB | Sulfosuccinimidyl 4-(p-maleimidophenyl)butyrate | 11.6 | Å |
| TMEA | Tr/s-(2-Maleimidoethyl)amine (Trifunctional) | 10.3 | Å |
| trifunctional | | | |
| crosslinker | | | |

The one or more attached linkers preferably comprise or the linkage preferably comprises 1,4-bis-maleimidobutane (BMB), bis-maleimide-PEG2 (BM-PEG2), BM-PEG3 or BM-PEG4. The one or more attached linkers preferably are or the linkage preferably is 1,4-bis-maleimidobutane (BMB), bis-maleimide-PEG2 (BM-PEG2), BM-PEG3 or BM-PEG4. BMB is 10.9 Å in length. BM-PEG2 is 14.7 Å in length. BM-PEG3 is 17.8 Å in length. BM-PEG4 is 20.9 Å in length. The NS3 helicase is preferably derived from a dengue virus (NS3-DV), a yellow fever virus (NS3-YFV), or a hepatitis C virus (NS3-HCV). The NS3 helicase is preferably derived from any of these viruses before it is modified in accordance with the invention. The NS3 helicase may be derived from any of the NS3 helicases shown in the table below. The NS3 helicase is preferably derived from HCV-JFH1 (NS3-HCV-JFH1).

| | TaxID | UniprotKB/ |
|---|---|---|
| Origin | (species) | Swiss-Prot |
| Dengue Virus (DV) | 408688 (12637) | Q2YHF0.1 |
| Yellow Fever | 11090 (11089) | P03314.1 |
| (YFV) | | |
| Hepatitis C virus | 333284 (11103) | Q9WMX2.3 |
| (NS3-HCV) | | |
| HCV-JFH1 | 356411 (11103) | Q99IB8.3 |

The NS3-DV is preferably the one described in Luo et al. (2008) The EMBO Journal, 27 (23), pp. 3209-3219. doi:https://doi.org/10.1038/emboj.2008.232. The NS3-YFV is preferably the one described in Wu et al. Journal of Virology, [online] 79 (16), pp. 10268-10277. doi:https://doi.org/10.1128/JVI.79.16.10268-10277.2005. The NS3-HCV is preferably the one described in Gu, M. and Rice, C. M. (2009) Proceedings of the National Academy of Sciences, 107 (2), pp. 521-528 (doi:https://doi.org/10.1073/pnas.0913380107). The NS3-HCV-JFH1 is preferably the one described in Zhou et al. (2017) Journal of Virology, 92 (1). doi:https://doi.org/10.1128/jvi.01253-17.

In NS3-DV, the two or more parts preferably comprise or are domains 2 and 3. In NS3-DV, the two amino acid residues are preferably in domains 2 and 3 respectively. In NS3-DV, the one or more attached linkers or linkage preferably connect or link domains 2 and 3. Domains 2 and 3 of NS3-DV are identified in Luo et al. (2008) The EMBO Journal, 27 (23), pp. 3209-3219. doi:https://doi.org/10.1038/emboj.2008.232.

In NS3-DV, the two or more parts or the two amino acid residues preferably comprise or preferably are positions 1199 and P438, positions I199 and M439, positions T223 and P438, or positions T223 and M439. In NS3-DV, the one or more attached linkers or the linkage preferably link positions I199 and P438, positions I199 and M439, positions T223 and P438, or positions T223 and M439. As discussed in more detail below, a cysteine or 4-azido-L-phenylalanine (Faz) may be introduced at one or both of these positions to facilitate linkage. In NS3-DV, the two or more parts or the two amino acid residues preferably comprise or preferably are or the one or more attached linkers or linkage preferably connect or link I199C/P438C, I199C/M439C, T223C/P438C, T223C/M439C, I199Faz/P438Faz, I199Faz/M439Faz, T223Faz/P438Faz, T223Faz/M439Faz, I199C/P438Faz, I199C/M439Faz, T223C/P438Faz, T223C/M439Faz, I199Faz/P438C, I199Faz/M439C, T223Faz/P438C, or T223C/M439C. Linking all the positions in this paragraph link domains 2 and 3 of NS3-DV.

In NS3-YFV, the two or more parts preferably comprise or are domains 1 and 3. In NS3-YFV, the two amino acid residues are preferably in domains 1 and 3 respectively. In NS3-YFV, the one or more attached linkers or linkage preferably connect or link domains 1 and 3. Domains 1 and 3 of NS3-YFV are identified in Wu et al. Journal of Virology, [online] 79 (16), pp. 10268-10277. doi:https://doi.org/10.1128/JVI.79.16.10268-10277.2005.

In NS3-YFV, the two or more parts or the two amino acid residues preferably comprise or preferably are positions Q68 and N361. In NS3-YFV, the one or more attached linkers or the linkage preferably link positions Q68 and N361. As discussed in more detail below, a cysteine or 4-azido-L-phenylalanine (Faz) may be introduced at one or both of these positions to facilitate linkage. In NS3-YFV, the two or more parts or the two amino acid residues preferably comprise or preferably are or the one or more attached linkers or linkage preferably connect or link Q68C/N361C, Q68Faz/N361Faz, Q68C/N361Faz, or Q68Faz/N361C. Linking the positions in this paragraph link domains 1 and 3 of NS3-YFV.

In NS3-HCV, the two or more parts preferably comprise or are domains 2 and 3. In NS3-HCV, the two amino acid residues are preferably in domains 2 and 3 respectively. In NS3-HCV, the one or more attached linkers or linkage preferably connect or link domains 2 and 3. Domains 2 and 3 of NS3-HCV are identified in Gu, M. and Rice, C. M. (2009) Proceedings of the National Academy of Sciences, 107 (2), pp. 521-528 (doi:https://doi.org/10.1073/pnas.0913380107). In NS3-HCV, the two or more parts or the two amino acid residues preferably comprise or preferably are positions G207 and G367. In NS3-HCV, the one or more attached linkers or linkage preferably connect or link positions G207 and G367. As discussed in more detail below, a cysteine or 4-azido-L-phenylalanine (Faz) may be introduced at one or both of these positions to facilitate linkage. In NS3-HCV, the two or more parts or the two amino acid residues preferably comprise or preferably are or the one or more attached linkers or linkage preferably connect or link G207C/G367C, G207Faz/G367Faz, G207C/G367Faz, or G207Faz/G367C. Linking the positions in this paragraph link domains 2 and 3 of NS3-HCV.

In NS3-HCV-JFH1, the two or more parts preferably comprise or are domains 2 and 3. In NS3-HCV-JFH1, the two amino acid residues are preferably in domains 2 and 3 respectively. In NS3-HCV-JFH1, the one or more attached linkers or linkage preferably connect or link domains 2 and 3. Domains 2 and 3 in NS3-HCV-JFH1 are identified in Zhou et al. (2017) Journal of Virology, 92(1). doi:https://doi.org/10.1128/jvi.01253-17.

In NS3-HCV-JFH1, the two or more parts or the two amino acid residues preferably comprise or preferably are positions G210 and G370. In NS3-DV, the one or more attached linkers or linkage preferably connect or link positions G210 and G370. As discussed in more detail below, a cysteine or 4-azido-L-phenylalanine (Faz) may be introduced at one or both of these positions to facilitate linkage. In NS3-HCV, the two or more parts or the two amino acid residues preferably comprise or preferably are or the one or more attached linkers or linkage preferably connect or link G210C/G370C, G210Faz/G370Faz, G210C/G370Faz, or G210Faz/G370C. Linking the positions in this paragraph link domains 2 and 3 of NS3-HCV-JFH1.

In a second embodiment, the invention provides a modified NS3 helicase comprising a polynucleotide binding domain, wherein two or more parts of the helicase are connected via one or more attached linkers to form a covalently closed structure around the polynucleotide binding domain, wherein the NS3 helicase is derived from a dengue virus (NS3-DV), a yellow fever virus (NS3-YFV), or a hepatitis C virus (NS3-HCV), and wherein the modified helicase retains its ability to control the movement of the polynucleotide.

In this second embodiment, the modified helicase may also be defined in any of the following ways:
The invention provides a modified NS3 helicase comprising a polynucleotide binding domain which comprises in at least one conformational state an opening through which a polynucleotide can unbind from the helicase, wherein the helicase is modified with one or more attached linkers such that it is capable of forming a covalently-closed structure around the polynucleotide, wherein the NS3 helicase is derived from a dengue virus (NS3-DV), a yellow fever virus (NS3-YFV), or a hepatitis C virus (NS3-HCV) and wherein the helicase retains its ability to control the movement of the polynucleotide.
The invention also provides a complex comprising (i) a NS3 helicase that comprises a polynucleotide binding domain, and (ii) a target polynucleotide bound to the polynucleotide binding domain, wherein two amino acid residues that are located in different structural domains on the surface of the NS3 helicase surrounding the polynucleotide binding domain are artificially covalently connected via a linkage between the two amino acid residues, such that the helicase has a covalently-closed structure, wherein the bound target polynucleotide is encircled by the covalently-closed structure and wherein the NS3 helicase is derived from a dengue virus (NS3-DV), a yellow fever virus (NS3-YFV), or a hepatitis C virus (NS3-HCV).
The invention also provides a modified NS3 helicase comprising a polynucleotide binding domain which comprises in at least one conformational state an opening through which a polynucleotide can unbind from the helicase, wherein the helicase is modified such that two or more parts of the helicase are covalently connected using one or more attached linkers and wherein the helicase retains its ability to control the movement of the polynucleotide, wherein the one or more attached linkers connect two amino acid residues that are located in different structural domains on the surface of the NS3 helicase surrounding the polynucleotide binding domain and wherein the NS3 helicase is derived from a dengue virus (NS3-DV), a yellow fever virus (NS3-YFV), or a hepatitis C virus (NS3-HCV). The at least one amino acid of the two amino acid residues is preferably substituted with cysteine, a non-natural amino acid or 4-azido-L-phenylalamine (Faz).

In the second embodiment, the one or more attached linkers or the linkage preferably have any of the lengths discussed above with reference to the first embodiment. The one or more attached linkers or the linkage in the second embodiment may be selected from any of the linkers in the table above. The one or more attached linkers or the linkage preferably comprise(s) or is/are 1,4-bis-maleimidobutane (BMB), bis-maleimide-PEG2 (BM-PEG2), BM-PEG3 or BM-PEG4.

The NS3-DV, NS3-YFV, or the NS3-HCV may be any of those discussed above with reference to the first embodiment. The NS3 helicase is preferably derived from HCV-JFH1 (NS3-HCV-JFH1). In NS3-DV, NS3-YFV, NS3-HCV, or NS3-HCV-JFH1, the two or more parts or the two amino acid residues preferably comprise or preferably are any of the positions discussed above with reference to the first embodiment. A cysteine or 4-azido-L-phenylalanine (Faz) may be introduced at one or both of these positions to facilitate linkage as described above with reference to the first embodiment.

A modified NS3 helicase of the invention is a useful tool for controlling the movement of a polynucleotide during Strand Sequencing. A problem which occurs in sequencing polynucleotides, particularly those of 100 nucleotides or more, is that the molecular motor which is controlling the movement of the polynucleotide may disengage from the polynucleotide. This allows the polynucleotide to be pulled through the pore rapidly and in an uncontrolled manner in the direction of the applied field. A modified NS3 helicase of the invention is less likely to unbind or disengage from the polynucleotide being sequenced. The modified NS3 helicase can provide increased read lengths of the polynucleotide as they control the movement of the polynucleotide through a pore. The ability to move an entire polynucleotide through a pore under the control of a modified NS3 helicase of the invention allows characteristics of the polynucleotide, such as its sequence, to be estimated with improved accuracy and speed over known methods. This becomes more important as strand lengths increase and molecular motors are required with improved processivity. A modified NS3 helicase of the invention is particularly effective in controlling the movement of target polynucleotides of 100 nucleotides or more, for example 500 nucleotides, 1000 nucleotides, 5000, 10000, 20000, 50000, 100000 or more. The modified NS3 helicase has the ability to control the movement of a polynucleotide. The ability of a helicase to control the movement of a polynucleotide can be assayed using any method known in the art. For instance, the helicase may be contacted with a polynucleotide and the position of the polynucleotide may be determined using standard methods. The ability of a modified NS3 helicase to control the movement of a polynucleotide is typically assayed in a pore system, such as the ones described below and, in particular, as described in the Examples.

A modified NS3 helicase of the invention may be isolated, substantially isolated, purified or substantially purified. A helicase is isolated or purified if it is completely free of any other components, such as lipids, polynucleotides, pore monomers or other proteins. A helicase is substantially isolated if it is mixed with carriers or diluents which will not interfere with its intended use. For instance, a helicase is substantially isolated or substantially purified if it is present in a form that comprises less than 10%, less than 5%, less than 2% or less than 1% of other components, such as lipids, polynucleotides, pore monomers or other proteins.

The modified NS3 helicase comprises a polynucleotide binding domain. A polynucleotide binding domain is the part of the helicase that is capable of binding to a polynucleotide. Polynucleotides are defined below. The ability of a domain to bind a polynucleotide can be determined using any method known in the art. The polynucleotide binding domains of known helicases have typically been identified in the art. The domain (with or without bound polynucleotide) may be identified using protein modelling, structure prediction, x-ray diffraction measurement of the protein in a crystalline state (Rupp B (2009). Biomolecular Crystallography: Principles, Practice and Application to Structural Biology. New York: Garland Science.), nuclear magnetic resonance (NMR) spectroscopy of the protein in solution (Mark Rance; Cavanagh, John; Wayne J. Fairbrother; Arthur W. Hunt III; Skelton, N Nicholas J. (2007). Protein NMR spectroscopy: principles and practice (2nd ed.). Boston: Academic Press.) or cryo-electron microscopy of the protein in a frozen-hydrated state (van Heel M, Gowen B, Matadeen R, Orlova E V, Finn R, Pape T, Cohen D, Stark H, Schmidt R, Schatz M, Patwardhan A (2000). “Single-particle electron cryo-microscopy: towards atomic resolution.”. Q Rev Biophys. 33:307-69. Structural information of proteins determined by above mentioned methods are publicly available from the protein bank (PDB) database.

Proteins, such as helicases, are dynamic structures which are in constant motion. The conformational space that a protein can explore has been described by an energy landscape, in which different conformations are populated based on their energies, and rates of interconversion are dependent on the energy barriers between states (Vinson, Science, 2009: 324(5924): 197). Helicases can therefore exist in several conformation states whether in isolation or controlling the movement of a polynucleotide. In at least one conformational state, the polynucleotide binding domain of an unmodified NS3 helicase for use in the invention typically comprises an opening through which a polynucleotide can unbind from the helicase. The opening may be present in all conformational states of the helicase, but does not have to be. For instance, in all conformational states, the polynucleotide binding domain may comprise an opening through which a polynucleotide can unbind from the helicase. Alternatively, in one or more conformational states of the helicase, the polynucleotide binding domain may comprise an opening through which a polynucleotide cannot unbind from the helicase because the opening is too small. In one or more conformational states of the helicase, the polynucleotide binding domain may not comprise an opening through which a polynucleotide can unbind from the helicase.

The polynucleotide binding domain preferably comprises in at least one conformational state an opening through which one or more internal nucleotides of the polynucleotide can unbind from the helicase. An internal nucleotide is a nucleotide which is not a terminal nucleotide in the polynucleotide. For example, it is not a 3′ terminal nucleotide or a 5′ terminal nucleotide. All nucleotides in a circular polynucleotide are internal nucleotides. Reducing or preventing the unbinding from one or more internal nucleotides in accordance with the invention is advantageous because it results in modified helicases that are capable of binding to one terminus of a polynucleotide, controlling the movement of most, if not all of, the polynucleotide and then unbinding at the other terminus. Such helicases are particularly helpful for Strand Sequencing. The ability of one or more internal nucleotide to unbind from the helicase may be determined by carrying out a comparative assay. For instance, the ability of a helicase to unbind from a control polynucleotide A is compared with its ability to unbind from the same polynucleotide but with a blocking group attached at the terminal nucleotides (polynucleotide B). The blocking group prevents any unbinding at the terminal nucleotide of strand B, and thus allows only internal unbinding of the helicase. Alternatively, the ability of a helicase to unbind from a circular polynucleotide may be assayed. Unbinding may be assayed as described above.

The opening may be a groove, pocket, or recess in the polynucleotide binding domain.

The presence of an opening through which a polynucleotide can unbind from the helicase can be determined using any method known in the art. The presence of an opening can be determined by measuring the ability of a helicase to unbind from a polynucleotide, and in particular from internal nucleotides of the polynucleotide, as discussed in more detail above. Openings in the polynucleotide domain can be identified using protein modelling, structure prediction, x-ray diffraction, NMR spectroscopy or cryo-electron microscopy as discussed above.

In accordance with the invention, the NS3 helicase is modified by two or more parts of the helicase being connected via one or more attached linkers to form a covalently closed structure. This may alternatively be defined as:
The helicase is modified with one or more attached linkers such that it is capable of forming a covalently closed structure around the polynucleotide.
Two amino acid residues that are located in different structural domains on the surface of the NS3 helicase surrounding the polynucleotide binding domain are artificially covalently connected via a linkage between the two amino acid residues, such that the helicase has a covalently closed structure.
The helicase is modified such that two or more parts of the helicase are covalently connected using one or more attached linkers.

Any number of two or more parts, such as 3, 4, 5 or more parts, may be connected. Preferred methods of connecting the two or more parts or two amino acids are discussed in more detail below.

The two or more parts or two amino acids can be located anywhere as long as they surround the polynucleotide binding domain or form a covalently closed structure around the polynucleotide binding domain. The two or more parts or two amino acids may be in the polynucleotide domain or the opening, but do not have to be. For instance, one, both or all of the two or more parts or two amino acids may be outside the polynucleotide binding domain, such as on different domain of the helicase.

At least one of the two or more parts or two amino acids preferably forms part of the opening, is adjacent to the opening or is near the opening. It is straightforward to identify parts or amino acids of the opening, such as amino acids within the opening, as described above. Parts or amino acids are adjacent to the opening if they are next to, but do not form part of the opening. For instance, an amino acid which is located next to an amino acid that forms part of the opening, but which itself does not form part of the opening is adjacent to the opening. In the context of the invention, “next to” may mean next to in the amino acid sequence of the helicase or next two in the three-dimensional structure of the helicase. A part or amino acid is typically near to the opening if it is less than 20 Å from an amino acid that forms part of the opening, such as less than 15 Å, less than 10 Å, less than 5 Å or less than 2 Å apart from an amino acid that forms part of the opening. A part or an amino acid is typically near to the opening if it is within 1, 2, 3, 4 or 5 amino acids of an amino acid that forms part of the opening in the amino acid sequence of the helicase. Such amino acids may be identified as discussed above. The two or more parts or two amino acids are typically on opposite sides of the opening.

The two or more parts or two amino acids are preferably on the surface of the helicase. It is straightforward to connect two or more parts or two amino acids on the surface as described in more detail below. The one or more attached linkers or linkage preferably covalently attach or connect surface parts or amino acids. Surface parts or amino acids may be determined using protein modelling, structure prediction, x-ray diffraction, NMR spectroscopy or cryo-electron microscopy as discussed above.

The modified NS3 helicase retains its ability to control the movement of a polynucleotide. This ability of the helicase is typically provided by its three-dimensional structure that is typically provided by its β-strands and α-helices. The □-helices and β-strands are typically connected by loop regions. In order to avoid affecting the ability of the helicase to control the movement of a polynucleotide, the two or more parts or two amino acids are preferably loop regions of the monomer. The one or more attached linkers or linkage preferably covalently attach or connect loop regions. The loop regions of specific helicases can be identified using methods known in the art, such as protein modelling, structure prediction, x-ray diffraction, NMR spectroscopy or cryo-electron microscopy as discussed above.

The NS3 helicase is modified to close the opening. If the opening is closed, the polynucleotide cannot unbind from the helicase through the opening. The helicase is more preferably modified such that it does not comprise the opening in any conformational state. If the opening is not present in any conformational state of the helicase, the polynucleotide cannot unbind from the helicase through the opening. The helicase is typically modified such that it is capable of forming a covalently closed structure around the polynucleotide. Once the covalently closed structure is bound to a polynucleotide, for instance at one end of the polynucleotide, it is capable of controlling the movement of the polynucleotide without unbinding until it reaches the other end.

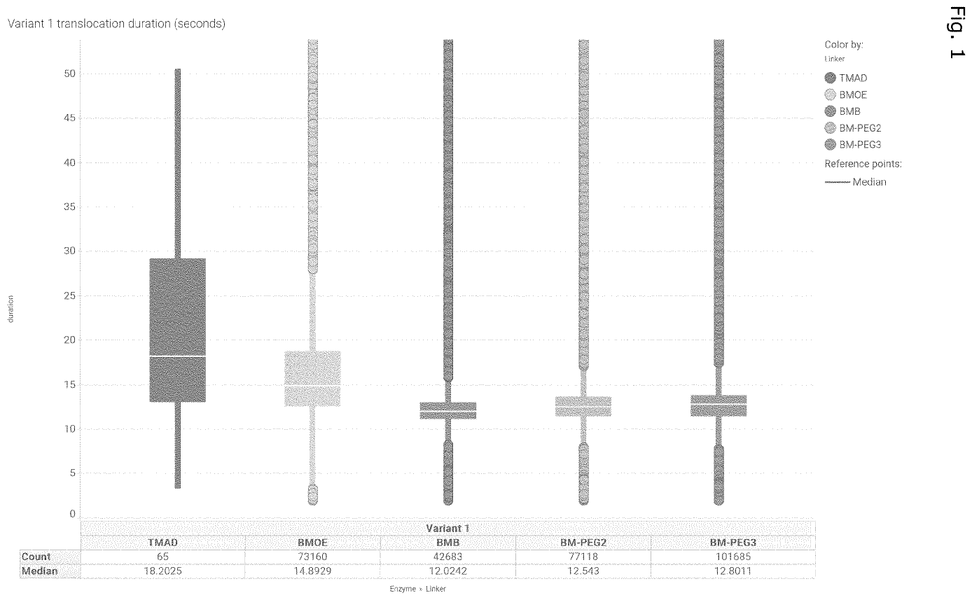

The two or more parts or two amino acids are preferably covalently attached. The two or more parts or two amino acids may be covalently attached using any method known in the art. The one or more attached linkers or the linkage preferably form a covalent attachment or connection. Covalent closure, attachment, connection, or linkage can occur via naturally occurring amino acids in the NS3 helicase, such as cysteines, threonines, serines, aspartates, asparagines, glutamates and glutamines. Naturally occurring amino acids may be modified to facilitate covalent linkage or attachment. For instance, the naturally occurring amino acids may be modified by acylation, phosphorylation, glycosylation or farnesylation. Other suitable modifications are known in the art. Modifications to naturally occurring amino acids may be post-translation modifications. The two or more parts or two amino acids may be attached via amino acids that have been introduced into the helicase sequence. The one or more attached linkers or linkage preferably connect or link amino acids that have been introduced into the helicase sequences. Such amino acids are preferably introduced by substitution. The introduced amino acid may be cysteine or a non-natural amino acid that facilitates attachment. Suitable non-natural amino acids include, but are not limited to, 4-azido-L-phenylalanine (Faz), any one of the amino acids numbered 1-71 included in FIG. 1 of Liu C. C. and Schultz P. G., Annu. Rev. Biochem., 2010, 79, 413-444 or any one of the amino acids listed below. The introduced amino acids may be modified as discussed above.

The modified NS3 helicase comprises one or more attachment linkers or linkage. Linker molecules are discussed in more detail above. One suitable method of connection is cysteine linkage. This is discussed in more detail below. Any number of attachment linkers may be used, such as two, three, four or more linkers. At least a part of the one or more attached linkers is preferably oriented such that it is not parallel to the polynucleotide when it is bound by the helicase. More preferably, all of the linkers are oriented in this manner. At least a part of the one or more attached linkers preferably crosses the opening in an orientation that is not parallel to the polynucleotide when it bound by the helicase. More preferably, all of the attached linkers cross the opening in this manner. In these embodiments, at least a part of the one or more attached linkers may be perpendicular to the polynucleotide. Such orientations effectively close the opening such that the polynucleotide cannot unbind from the helicase through the opening.

Each attached linker may have two or more functional ends, such as two, three or four functional ends. Suitable configurations of ends in linkers are well known in the art.

Both or all ends of the one or more attached linkers are preferably covalently attached to the helicase. If both or all ends are covalently attached, the one or more linkers permanently connect the two or more parts.

The helicase is preferably modified to facilitate the attachment of the one or more attached linkers. At least one of the two or more parts or two amino acids is preferably modified to facilitate the attachment of the one or more attached linkers. Any modification may be made.

The attached linkers may be attached to one or more reactive cysteine residues, reactive lysine residues or non-natural amino acids in the two or more parts or two amino acids. The non-natural amino acid may be any of those discussed above. The non-natural amino acid is preferably 4-azido-L-phenylalanine (Faz). At least one amino acid in the two or more parts or two amino acids is preferably substituted with cysteine or a non-natural amino acid, such as Faz.

The one or more linkers are preferably amino acid sequences and/or chemical crosslinkers. Suitable amino acid linkers, such as peptide linkers, are known in the art. The length, flexibility and hydrophilicity of the amino acid or peptide linker are typically designed such that closes the opening, but does not to disturb the functions of the helicase. Preferred flexible peptide linkers are stretches of 2 to 20, such as 4, 6, 8, 10 or 16, serine and/or glycine amino acids. More preferred flexible linkers include (SG)₁, (SG)₂, (SG)₃, (SG)₄, (SG)₅, (SG)₈, (SG)₁₀, (SG)₁₅ or (SG)₂₀ wherein S is serine and G is glycine. Preferred rigid linkers are stretches of 2 to 30, such as 4, 6, 8, 16 or 24, proline amino acids. More preferred rigid linkers include (P) 12 wherein P is proline. The amino acid sequence of a linker preferably comprises a polynucleotide binding moiety. Such moieties and the advantages associated with their use are discussed below.

Suitable chemical crosslinkers are well-known in the art. Suitable chemical crosslinkers include, but are not limited to, those including the following functional groups: maleimide, active esters, succinimide, azide, alkyne (such as dibenzocyclooctynol (DIBO or DBCO), difluoro cycloalkynes and linear alkynes), phosphine (such as those used in traceless and non-traceless Staudinger ligations), haloacetyl (such as iodoacetamide), phosgene type reagents, sulfonyl chloride reagents, isothiocyanates, acyl halides, hydrazines, disulphides, vinyl sulfones, aziridines and photoreactive reagents (such as aryl azides, diaziridines).

Reactions between amino acids and functional groups may be spontaneous, such as cysteine/maleimide, or may require external reagents, such as Cu(I) for linking azide and linear alkynes.

Linkers can comprise any molecule that stretches across the distance required. Examples of linear molecules, include but are not limited to, are polyethyleneglycols (PEGs), polypeptides, polysaccharides, deoxyribonucleic acid (DNA), peptide nucleic acid (PNA), threose nucleic acid (TNA), glycerol nucleic acid (GNA), saturated and unsaturated hydrocarbons, polyamides.

These linkers may be inert or reactive, in particular they may be chemically cleavable at a defined position, or may be themselves modified with a fluorophore or ligand. The linker is preferably resistant to dithiothreitol (DTT).

Preferred crosslinkers are described above with. The one or more linkers may be cleavable. This is discussed in more detail below.

The invention may use two different linkers that are specific for each other. One of the linkers is attached to one part of the helicase and the other is attached to another part. The linkers should react to form a modified NS3 helicase of the invention. The invention may use the hybridization linkers described in International Application No. PCT/GB 10/000132 (published as WO 2010/086602). Any of the specific linkers disclosed in International Application No. PCT/GB10/000132 (published as WO 2010/086602) may be used in accordance with the invention.

The one or more attached linkers may be labelled. Suitable labels include, but are not limited to, fluorescent molecules (such as Cy3 or AlexaFluor®555), radioisotopes, e.g. ¹²⁵I, ³⁵S, enzymes, antibodies, antigens, polynucleotides, and ligands such as biotin. Such labels allow the amount of linker to be quantified. The label could also be a cleavable purification tag, such as biotin, or a specific sequence to show up in an identification method, such as a peptide that is not present in the protein itself, but that is released by trypsin digestion.

A preferred method of connecting is via cysteine linkage. This can be mediated by a bi-functional chemical crosslinker or by an amino acid linker with a terminal presented cysteine residue. Linkage can occur via natural cysteines in the helicase. Alternatively, cysteines can be introduced into the helicase, preferably by substitution. Examples of this are described above. One drawback of bi-functional linkers is the requirement of the helicase to contain no further surface accessible cysteine residues if attachment at specific sites is preferred, as binding of the bi-functional linker to surface accessible cysteine residues may be difficult to control and may affect substrate binding or activity. If the helicase does contain several accessible cysteine residues, modification of the helicase may be required to remove them while ensuring the modifications do not affect the folding or activity of the helicase. This is discussed in International Application No. PCT/GB10/000133 (published as WO 2010/086603). The reactivity of cysteine residues may be enhanced by modification of the adjacent residues, for example on a peptide linker. For instance, the basic groups of flanking arginine, histidine or lysine residues will change the pKa of the cysteines thiol group to that of the more reactive S− group. The reactivity of cysteine residues may be protected by thiol protective groups such as 5,5′-dithiobis-(2-nitrobenzoic acid) (dTNB). These may be reacted with one or more cysteine residues of the helicase before a linker is attached. Selective deprotection of surface accessible cysteines may be possible using reducing reagents immobilized on beads (for example immobilized tris(2-carboxyethyl) phosphine, TCEP). Cysteine linkage is discussed in more detail below.

Another preferred method of linkage is via 4-azido-L-phenylalanine (Faz) linkage. This can be mediated by a bi-functional chemical linker or by a polypeptide linker with a terminal presented Faz residue. The one or more Faz residues have preferably been introduced to the helicase by substitution. Examples of this are shown above.

### Method of Increasing Helicase Speed

In a third embodiment, the invention provides a method of increasing the speed at which a helicase is capable of controlling the movement of the polynucleotide, wherein the helicase comprises a polynucleotide binding domain, the method comprising modifying the helicase by connecting two or more parts of the helicase via one or more attached linkers greater than about 8.0 angstroms (Å) in length to form a covalently closed structure around the polynucleotide binding domain. The method of the third embodiment may also be defined in any of the following ways:
The invention also provides a method of increasing the speed at which a helicase is capable of controlling the movement of a polynucleotide, wherein the helicase comprises a polynucleotide binding domain which comprises in at least one conformational state an opening through which a polynucleotide can unbind from the helicase, the method comprising modifying the helicase with one or more attached linkers greater than about 8.0 angstroms (Å) in length such that it is capable of forming a covalently closed structure around the polynucleotide.
The invention also provides a method of increasing the speed at which a complex is capable of controlling the movement of a target polynucleotide, wherein the complex comprises (i) a helicase that comprises a polynucleotide binding domain, and (ii) a target polynucleotide bound to the polynucleotide binding domain, the method comprising artificially covalently connecting two amino acid residues that are located in different structural domains on the surface of the helicase surrounding the polynucleotide binding domain via a linkage greater than about 8.0 angstroms (Å) in length between the two amino acid residues, such that the helicase has a covalently closed structure, and wherein the bound target polynucleotide is encircled by the covalently-closed structure.
The invention also provides a method of increasing the speed at which a helicase is capable of controlling the movement of a polynucleotide, wherein the helicase comprises a polynucleotide binding domain which comprises in at least one conformational state an opening through which a polynucleotide can unbind from the helicase, the method comprising modifying the helicase such that two or more parts of the helicase are covalently connected using one or more attached linkers greater than about 8.0 angstroms (Å) in length, wherein the helicase retains its ability to control the movement of the polynucleotide, and wherein the one or more attached linkers connect two amino acid residues that are located in different structural domains on the surface of the helicase surrounding the polynucleotide binding domain. At least one amino acid of the two amino acid residues is or the two amino acids are preferably substituted with cysteine, a non-natural amino acid or 4-azido-L-phenylalamine (Faz).

In all methods, the modified helicase typically retains its ability to control the movement of the polynucleotide. This can be measured as discussed above.

The length of the one or more attached linkers or the linkage is preferably greater than about 8.5 Å, greater than about 9.0 Å, greater than about 9.5 Å, greater than about 10.0 Å, greater than about 10.5 Å, greater than about 11.0 Å, greater than about 11.5 Å, greater than about 12.0 Å, greater than about 12.5 Å, greater than about 13.0 Å, greater than about 13.5 Å, greater than about 14.0 Å, greater than about 14.5 Å, greater than about 15.0 Å, greater than about 15.5 Å, greater than about 16.0 Å, or greater than about 16.5 Å, greater than about 17.0 Å, greater than about 17.5 Å, greater than about 18.0 Å, greater than about 18.5 Å, greater than about 19.0 Å, greater than about 19.5 Å, greater than about 20.0 Å, greater than about 20.5 Å, greater than about 20.9 Å, greater than about 21.0 Å, greater than about 21.5 Å, greater than about 22.0 Å, greater than about 22.5 Å, greater than about 23.0 Å, greater than about 23.5 Å, greater than about 24.0 Å, greater than about 24.5 Å, or greater than about 25.0 Å in length.

The length of the one or more attached linkers or the linkage is preferably at least about 10.0 Å in length. The length of the one or more attached linkers or the linkage is preferably at least about 10.5 Å in length, at least about 11.0 Å in length, at least about 11.5 Å in length, at least about 12.0 Å in length, at least about 12.5 Å in length, at least about 13.0 Å in length, at least about 13.5 Å in length, at least about 14.0 Å in length, at least about 14.5 Å in length, at least about 15.0 Å in length, at least about 15.5 Å in length, at least about 16.0 Å in length, at least about 16.5 Å in length, at least about 17.0 Å in length, at least about 17.5 Å in length, at least about 18.0 Å in length, at least about 18.5 Å in length, at least about 19.0 Å in length, at least about 19.5 Å in length, at least about 20.0 Å in length, at least about 20.5 Å in length, at least about 20.9 Å, at least about 21.0 Å in length, at least about 21.5 Å in length, at least about 22.0 Å in length, at least about 22.5 Å in length, at least about 23.0 Å in length, at least about 23.5 Å in length, at least about 24.0 Å in length, at least about 24.5 Å in length, or at least about 25.0 Å in length.

The length of the one or more attached linkers or the linkage is preferably from about 8.5 Å to about 30.0 Å in length. The length of the one or more attached linkers or the linkage is preferably from about 9.0 Å to about 25.0 Å in length, from about 9.5 Å to about 22.0 Å in length, from about 9.75 Å to about 20.9 Å, from about 10.0 Å to about 20.0 Å in length, from about 10.5 Å to about 18.0 Å in length, from about 10.9 Å to about 18.0 Å in length, from about 10.5 Å to about 17.8 Å in length, or from about 10.9 Å to about 17.8 Å in length.

The length of the one or more attached linkers or the linkage is preferably about 10.9 Å, about 14.7 Å, about 17.8 Å, or about 20.9 Å in length. The length of the one or more attached linkers or the linkage is preferably about 10.9 Å, about 14.7 Å, or about 17.8 Å in length.

The one or more attached linkers or the linkage in the third embodiment may be selected from any of the linkers in the table above.

The one or more attached linkers or the linkage preferably comprise(s) or is/are 1,4-bis-maleimidobutane (BMB), bis-maleimide-PEG2 (BM-PEG2), BM-PEG3 or BM-PEG4. BMB is 10.9 Å in length. BM-PEG2 is 14.7 Å in length. BM-PEG3 is 17.8 Å in length. BM-PEG4 is 20.9 Å in length.

In a fourth embodiment, the invention provides a method of increasing the speed at which a modified helicase is capable of controlling the movement of the polynucleotide, wherein the modified helicase comprises a polynucleotide binding domain, wherein two or more parts of the helicase are connected via one or more attached linkers to form a covalently closed structure around the polynucleotide binding domain, the method comprising increasing the length of the one or more attached linkers. The method of the fourth embodiment may also be defined in any of the following ways:
The invention also provides a method of increasing the speed at which a modified helicase is capable of controlling the movement of a polynucleotide, wherein the modified helicase comprises a polynucleotide binding domain which comprises in at least one conformational state an opening through which a polynucleotide can unbind from the helicase, and wherein the helicase is modified with one or more attached linkers such that it is capable of forming a covalently closed structure around the polynucleotide, the method comprising increasing the length of the one or more attached linkers.
The invention also provides a method of increasing the speed at which a complex is capable of controlling the movement of a target polynucleotide, wherein the complex comprises (i) a helicase that comprises a polynucleotide binding domain, and (ii) a target polynucleotide bound to the polynucleotide binding domain, wherein two amino acid residues that are located in different structural domains on the surface of the helicase surrounding the polynucleotide binding domain are artificially covalently connected via a linkage between the two amino acid residues, such that the helicase has a covalently closed structure, and wherein the bound target polynucleotide is encircled by the covalently-closed structure, the method comprising increasing the length of the linkage.
The invention also provides a method of increasing the speed at which a modified helicase is capable of controlling the movement of a polynucleotide, wherein the modified helicase comprises a polynucleotide binding domain which comprises in at least one conformational state an opening through which a polynucleotide can unbind from the helicase, wherein the helicase is modified such that two or more parts of the helicase are covalently connected using one or more attached linkers and wherein the helicase retains its ability to control the movement of the polynucleotide, and wherein the one or more attached linkers connect two amino acid residues that are located in different structural domains on the surface of the helicase surrounding the polynucleotide binding domain, the method comprising increasing the length of the one or more attached linkers. At least one amino acid of the two amino acid residues is or the two amino acids are preferably substituted with cysteine, a non-natural amino acid or 4-azido-L-phenylalamine (Faz).

In all methods, the modified helicase typically retains its ability to control the movement of the polynucleotide. This can be measured as discussed above.

In the fourth embodiment, the method comprises increasing the length of the one or more attached linkers or the linkage. The length of the one or more attached linkers or the linkage is preferably increased to greater than about 8 angstroms (Å) in length. The length of the one or more attached linkers or the linkage is preferably increased to greater than about 8.5 Å, greater than about 9.0 Å, greater than about 9.5 Å, greater than about 10.0 Å, greater than about 10.5 Å, greater than about 11.0 Å, greater than about 11.5 Å, greater than about 12.0 Å, greater than about 12.5 Å, greater than about 13.0 Å, greater than about 13.5 Å, greater than about 14.0 Å, greater than about 14.5 Å, greater than about 15.0 Å, greater than about 15.5 Å, greater than about 16.0 Å, or greater than about 16.5 Å, greater than about 17.0 Å, greater than about 17.5 Å, greater than about 18.0 Å, greater than about 18.5 Å, greater than about 19.0 Å, greater than about 19.5 Å, greater than about 20.0 Å, greater than about 20.5 Å, greater than about 20.9 Å, greater than about 21.0 Å, greater than about 21.5 Å, greater than about 22.0 Å, greater than about 22.5 Å, greater than about 23.0 Å, greater than about 23.5 Å, greater than about 24.0 Å, greater than about 24.5 Å, or greater than about 25.0 Å in length.

The length of the one or more attached linkers or the linkage is preferably increased to at least about 10.0 Å in length. The length of the one or more attached linkers or the linkage is preferably increased to at least about 10.5 Å in length, at least about 11.0 Å in length, at least about 11.5 Å in length, at least about 12.0 Å in length, at least about 12.5 Å in length, at least about 13.0 Å in length, at least about 13.5 Å in length, at least about 14.0 Å in length, at least about 14.5 Å in length, at least about 15.0 Å in length, at least about 15.5 Å in length, at least about 16.0 Å in length, at least about 16.5 Å in length, at least about 17.0 Å in length, at least about 17.5 Å in length, at least about 18.0 Å in length, at least about 18.5 Å in length, at least about 19.0 Å in length, at least about 19.5 Å in length, at least about 20.0 Å in length, at least about 20.5 Å in length, at least about 20.9 Å in length, at least about 21.0 Å in length, at least about 21.5 Å in length, at least about 22.0 Å in length, at least about 22.5 Å in length, at least about 23.0 Å in length, at least about 23.5 Å in length, at least about 24.0 Å in length, at least about 24.5 Å in length, or at least about 25.0 Å in length.

The length of the one or more attached linkers or the linkage is preferably increased to from about 8.5 Å to about 30.0 Å in length. The length of the one or more attached linkers or the linkage is preferably increased to from about 9.0 Å to about 25.0 Å in length, from about 9.5 Å to about 22.0 Å in length, from about 9.75 Å to about 20.9 Å in length, from about 10.0 Å to about 20.0 Å in length, from about 10.5 Å to about 18.0 Å in length, from about 10.9 Å to about 18.0 Å in length, from about 10.5 Å to about 17.8 Å in length, or from about 10.9 Å to about 17.8 Å in length.

The length of the one or more attached linkers or the linkage is preferably increased to about 10.9 Å, about 14.7 Å, about 17.8 Å, or about 20.9 Å in length. The length of the one or more attached linkers or the linkage is preferably increased to about 10.9 Å, about 14.7 Å, or about 17.8 Å in length.

The one or more attached linkers or the linkage in the fourth embodiment may be selected from any of the linkers in the table above.

The length of the one or more attached linkers or the linkage is preferably increased by replacing the one or more attached linkers or linkage with 1,4-bis-maleimidobutane (BMB), bis-maleimide-PEG2 (BM-PEG2), BM-PEG3 or BM-PEG4. BMB is 10.9 Å in length. BM-PEG2 is 14.7 Å in length. BM-PEG3 is 17.8 Å in length. BM-PEG4 is 20.9 Å in length.

In both the third and fourth embodiments, the speed at which the modified helicase is capable of controlling the movement of the polynucleotide is increased at the end of the method. The speed is increased compared with speed of helicase or the modified helicase before the method is carried. Speed is typically determined by measuring the duration of the polynucleotide within a pore, such as any of the pores described below, when movement of the polynucleotide through the pore is controlled by the modified helicase. From this, it is possible to calculate the speed of the polynucleotide moving through the pore. Speed can also be measured by using a polynucleotide of a known length and measuring its translocation time through a pore. The strand may be mapped back to a reference to make sure the helicase is actively moving over the strand. Speed is calculated by dividing the length by the duration.

In both the third and fourth embodiments, the speed at which the helicase or modified helicase is capable of controlling the movement of the polynucleotide may be increased by any amount. The speed is preferably increased by at least about 1%. The speed is preferably increased by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%. The speed is preferably increase at least about 2-fold, such as by at least about 3-fold, at least about 4-fold, at least about 5-fold, at least about 6-fold, at least about 7-fold, at least about 8-fold, at least about 9-fold, or at least about 10-fold.

The method of third embodiment or the fourth embodiment preferably comprises measuring the speed at which the modified helicase is capable of controlling the movement of the polynucleotide and confirming the speed is increased. This can be done as described in the Examples.

The method of third embodiment or the fourth embodiment may increase the speed of any helicase or modified helicase comprising a polynucleotide binding domain which comprises in at least one conformational state an opening through which a polynucleotide can unbind from the helicase. Helicases are often known as translocases and the two terms may be used interchangeably. The helicase or modified helicase may be any of those disclosed in WO 2014/013260 (incorporated herein by reference in its entirety).

Suitable helicases are well-known in the art (M. E. Fairman-Williams et al., Curr. Opin. Struct Biol., 2010, 20 (3), 313-324, T. M. Lohman et al., Nature Reviews Molecular Cell Biology, 2008, 9, 391-401).

The helicase or modified helicase is preferably a member of superfamily 1 or superfamily 2. The helicase or modified helicase is more preferably a member of one of the following families: Pif1-like, Upf1-like, UvrD/Rep, Ski-like, Rad3/XPD, NS3/NPH-II, DEAD, DEAH/RHA, RecG-like, RecQ-like, T1R-like, Swi/Snf-like and Rig-I-like. The first three of those families are in superfamily 1 and the second ten families are in superfamily 2. The helicase or modified helicase is more preferably a member of one of the following subfamilies: Dda, RecD, Upf1 (RNA), PcrA, Rep, UvrD, Hel308, Mtr4 (RNA), XPD, NS3 (RNA), Mss116 (RNA), Prp43 (RNA), RecG, RecQ, T1R, RapA and Hef (RNA). The first five of those subfamilies are in superfamily 1 and the second eleven subfamilies are in superfamily 2. Members of the Upf1, Mtr4, NS3, Mss116, Prp43 and Hef subfamilies are RNA helicases. Members of the remaining subfamilies are DNA helicases. The helicase is most preferably a modified NS3 helicase. The NS3 helicase may be any of those described above.

The helicase or modified helicase may be a multimeric or oligomeric helicase. In other words, the helicase or modified helicase may need to form a multimer or an oligomer, such as a dimer, to function. In such embodiments, the two or more parts cannot be on different monomers. The helicase or modified helicase is preferably monomeric. In other words, the helicase or modified helicase preferably does not need to form a multimer or an oligomer, such as a dimer, to function. Dda, Hel308, RecD, TraI and XPD helicases are all monomeric helicases. Methods for determining whether or not a helicase or modified helicase is oligomeric/multimeric or monomeric are known in the art. For instance, the kinetics of radiolabelled or fluorescently labelled polynucleotide unwinding using the helicase or modified helicase can be examined. Alternatively, the helicase or modified helicase can be analysed using size exclusion chromatography.

Monomeric helicases may comprise several domains attached together. For instance, TraI helicases and TraI subgroup helicases may contain two RecD helicase domains, a relaxase domain and a C-terminal domain. The domains typically form a monomeric helicase that is capable of functioning without forming oligomers. The two or more parts may be present on the same or different domains of a monomeric helicase or monomeric modified helicase.

The helicase or modified helicase is preferably capable of binding to the target polynucleotide at an internal nucleotide and/or a terminal nucleotide. Such helicase are disclosed in WO 2014/013260 (incorporated herein by reference in its entirety).

The method may increase the speed of any one of the Dda helicases or modified Dda helicases disclosed in WO 2015/055981 (incorporated herein by reference in its entirety).

The method may increase the speed of any one of the Hel308 helicases or modified Hel308 helicases disclosed in WO 2014/013260 (incorporated herein by reference in its entirety). This includes modified versions of the Hel308 helicases shown in Table 1 of WO 2014/013260 (incorporated herein by reference in its entirety).

The method may increase the speed of any one of the RecD helicases or modified RecD helicases disclosed in WO 2014/013260 (incorporated herein by reference in its entirety). This includes the TraI helicases and TraI subgroup helicases or modified TraI helicases and TraI subgroup helicases disclosed in WO 2014/013260 (incorporated herein by reference in its entirety). This also includes modified versions of the TraI helicases or TraI subgroup helicases shown in Table 3 of WO 2014/013260 (incorporated herein by reference in its entirety).

The method may increase the speed of any one of the XPD helicases or modified XPD helicases disclosed in WO 2014/013260 (incorporated herein by reference in its entirety).

The helicase or modified helicase used in the method of the invention may comprise or consist of any of the variant sequences disclosed in WO 2014/013260 (incorporated herein by reference in its entirety).

The invention also provides a modified helicase which is capable of controlling the movement of the polynucleotide with an increased speed produced using a method of the invention. If produced using the method of the third embodiment, the modified helicase comprises one or more attached linkers or a linkage greater than about 8.0 angstroms (Å) in length. The length may be any of those discussed above. If produced using the method of the fourth embodiment, the modified helicase comprises one or more attached linkers or a linkage with an increased length. The length is increased compared with the length of the one or more attached linkers or linkage in the modified helicase before the method of the invention is carried. The increased length may be any of those described above. The speed of the modified helicase of the invention may be increased by any of the % or fold amounts discussed above with reference to the methods of the invention.

All subsequent references to a “modified helicase of the invention” include a modified NS3 helicase of the invention and a modified helicase of the invention which is capable of controlling the movement of the polynucleotide with an increased speed produced using a method of the invention. The modified NS3 helicase of the invention may be a modified NS3 helicase of the first embodiment or the second embodiment. The modified helicase with increased speed may be produced using a method of the third embodiment or the fourth embodiment.

The invention also provides a series of two or more helicases attached to a polynucleotide, wherein at least one of the two or more helicases is a modified helicase of the invention. The series may comprise any number of two or more helicases, such as 2, 3, 4, 5, 6, 7, 8, 9, 10 or more helicases. All of the two or more helicases are preferably modified helicases of the invention. Polynucleotides are defined below with reference to the methods of the invention.

### Constructs

The invention also provides a construct comprising a modified helicase of the invention and an additional polynucleotide binding moiety, wherein the modified helicase is attached to the polynucleotide binding moiety and the construct has the ability to control the movement of a polynucleotide.

The modified helicase is attached to the additional polynucleotide binding moiety. The construct is artificial or non-natural.

A construct of the invention is a useful tool for controlling the movement of a polynucleotide during Strand Sequencing. A construct of the invention is even less likely than a modified helicase of the invention to disengage from the polynucleotide being sequenced. The construct can provide even greater read lengths of the polynucleotide as it controls the translocation of the polynucleotide through a pore.

A targeted construct that binds to a specific polynucleotide sequence can also be designed. As discussed in more detail below, the polynucleotide binding moiety may bind to a specific polynucleotide sequence and thereby target the modified helicase portion of the construct to the specific sequence.

The construct has the ability to control the movement of a polynucleotide. This can be determined as discussed above.

A construct of the invention may be isolated, substantially isolated, purified or substantially purified. A construct is isolated or purified if it is completely free of any other components, such as lipids, polynucleotides or pore monomers. A construct is substantially isolated if it is mixed with carriers or diluents which will not interfere with its intended use. For instance, a construct is substantially isolated or substantially purified if it is present in a form that comprises less than 10%, less than 5%, less than 2% or less than 1% of other components, such as lipids, polynucleotides or pore monomers.

The modified helicase of the invention is preferably covalently attached to the additional polynucleotide binding moiety. The modified helicase of the invention may be attached to the moiety at more than one, such as two or three, points.

The modified helicase can be covalently attached to the moiety using any method known in the art. Suitable methods are discussed above.

The modified helicase and moiety may be produced separately and then attached together. The two components may be attached in any configuration. For instance, they may be attached via their terminal (i.e. amino or carboxy terminal) amino acids. Suitable configurations include, but are not limited to, the amino terminus of the moiety being attached to the carboxy terminus of the modified helicase and vice versa. Alternatively, the two components may be attached via amino acids within their sequences. For instance, the moiety may be attached to one or more amino acids in a loop region of the modified helicase. In a preferred embodiment, terminal amino acids of the moiety are attached to one or more amino acids in the loop region of a modified helicase.

In a preferred embodiment, the modified helicase is chemically attached to the moiety, for instance via one or more linker molecules as discussed above. In another preferred embodiment, the modified helicase is genetically fused to the moiety. A modified helicase is genetically fused to a moiety if the whole construct is expressed from a single polynucleotide sequence. The coding sequences of the modified helicase and moiety may be combined in any way to form a single polynucleotide sequence encoding the construct. Genetic fusion of a pore to a nucleic acid binding protein is discussed in International Application No. PCT/GB09/001679 (published as WO 2010/004265).

The modified helicase and moiety may be genetically fused in any configuration. The modified helicase and moiety may be fused via their terminal amino acids. For instance, the amino terminus of the moiety may be fused to the carboxy terminus of the modified helicase and vice versa. The amino acid sequence of the moiety is preferably added in frame into the amino acid sequence of the modified helicase. In other words, the moiety is preferably inserted within the sequence of the modified helicase. In such embodiments, the modified helicase and moiety are typically attached at two points, i.e. via the amino and carboxy terminal amino acids of the moiety. If the moiety is inserted within the sequence of the modified helicase, it is preferred that the amino and carboxy terminal amino acids of the moiety are in close proximity and are each attached to adjacent amino acids in the sequence of the modified helicase or variant thereof. In a preferred embodiment, the moiety is inserted into a loop region of the modified helicase.

The construct retains the ability of the modified helicase to control the movement of a polynucleotide. This ability of the modified helicase is typically provided by its three-dimensional structure that is typically provided by its β-strands and α-helices. The □-helices and β-strands are typically connected by loop regions. In order to avoid affecting the ability of the modified helicase to control the movement of a polynucleotide, the moiety is preferably genetically fused to either end of the modified helicase or inserted into a surface-exposed loop region of the modified helicase. The loop regions of specific modified helicases can be identified using methods known in the art. In the Hel308 embodiments of the invention, the moiety is preferably not genetically fused to any of the α-helixes.

The modified helicase may be attached directly to the moiety. The modified helicase is preferably attached to the moiety using one or more, such as two or three, linkers as discussed above. The one or more linkers may be designed to constrain the mobility of the moiety. The modified helicase and/or the moiety may be modified to facilitate attachment of the one or more linker as discussed above.

Cleavable linkers can be used as an aid to separation of constructs from non-attached components and can be used to further control the synthesis reaction. For example, a hetero-bifunctional linker may react with the modified helicase, but not the moiety. If the free end of the linker can be used to bind the modified helicase protein to a surface, the unreacted modified helicases from the first reaction can be removed from the mixture. Subsequently, the linker can be cleaved to expose a group that reacts with the moiety. In addition, by following this sequence of linkage reactions, conditions may be optimised first for the reaction to the modified helicase, then for the reaction to the moiety after cleavage of the linker. The second reaction would also be much more directed towards the correct site of reaction with the moiety because the linker would be confined to the region to which it is already attached.

The modified helicase may be covalently attached to the bifunctional crosslinker before the modified helicase/crosslinker complex is covalently attached to the moiety. Alternatively, the moiety may be covalently attached to the bifunctional crosslinker before the bifunctional crosslinker/moiety complex is attached to the modified helicase. The modified helicase and moiety may be covalently attached to the chemical crosslinker at the same time.

Preferred methods of attaching the modified helicase to the moiety are cysteine linkage and Faz linkage as described above. In a preferred embodiment, a reactive cysteine is presented on a peptide linker that is genetically attached to the moiety. This means that additional modifications will not necessarily be needed to remove other accessible cysteine residues from the moiety. Cross-linkage of modified helicases or moieties to themselves may be prevented by keeping the concentration of linker in a vast excess of the modified helicase and/or moiety. Alternatively, a “lock and key” arrangement may be used in which two linkers are used. Only one end of each linker may react together to form a longer linker and the other ends of the linker each react with a different part of the construct (i.e. modified helicase or moiety). This is discussed in more detail below.

The site of attachment is selected such that, when the construct is contacted with a polynucleotide, both the modified helicase and the moiety can bind to the polynucleotide and control its movement.

Attachment can be facilitated using the polynucleotide binding activities of the modified helicase and the moiety. For instance, complementary polynucleotides can be used to bring the modified helicase and moiety together as they hybridize. The modified helicase can be bound to one polynucleotide and the moiety can be bound to the complementary polynucleotide. The two polynucleotides can then be allowed to hybridise to each other. This will bring the modified helicase into close contact with the moiety, making the linking reaction more efficient. This is especially helpful for attaching two or more modified helicases in the correct orientation for controlling movement of a target polynucleotide.

Tags can be added to the construct to make purification of the construct easier. These tags can then be chemically or enzymatically cleaved off if their removal is necessary. Fluorophores or chromophores can also be included, and these could also be cleavable.

A simple way to purify the construct is to include a different purification tag on each protein (i.e. the modified helicase and the moiety), such as a hexa-His-tag and a Strep-Tag®. If the two proteins are different from one another, this method is particularly useful. The use of two tags enables only the species with both tags to be purified easily.

If the two proteins do not have two different tags, other methods may be used. For instance, proteins with free surface cysteines or proteins with linkers attached that have not reacted to form a construct could be removed, for instance using an iodoacetamide resin for maleimide linkers.

Constructs of the invention can also be purified from unreacted proteins on the basis of a different DNA processivity property. In particular, a construct of the invention can be purified from unreacted proteins on the basis of an increased affinity for a polynucleotide, a reduced likelihood of disengaging from a polynucleotide once bound and/or an increased read length of a polynucleotide as it controls the translocation of the polynucleotide through a pore.

A targeted construct that binds to a specific polynucleotide sequence can also be designed. As discussed in more detail below, the polynucleotide binding moiety may bind to a specific polynucleotide sequence and thereby target the modified helicase portion of the construct to the specific sequence.

The constructs of the invention comprise a polynucleotide binding moiety. Polynucleotides are defined below with reference to the methods of the invention. A polynucleotide binding moiety is a polypeptide that is capable of binding to a polynucleotide. The moiety is preferably capable of specific binding to a defined polynucleotide sequence. In other words, the moiety preferably binds to a specific polynucleotide sequence, but displays at least 10-fold less binding to different sequences or more preferably at least 100 fold less binding to different sequences or most preferably at least 1000 fold less binding to different sequences. The different sequence may be a random sequence. In some embodiments, the moiety binds to a specific polynucleotide sequence, but binding to different sequences cannot be measured. Moieties that bind to specific sequences can be used to design constructs that are targeted to such sequences.

The moiety typically interacts with and modifies at least one property of a polynucleotide. The moiety may modify the polynucleotide by cleaving it to form individual nucleotides or shorter chains of nucleotides, such as di- or trinucleotides. The moiety may modify the polynucleotide by orienting it or moving it to a specific position, i.e. controlling its movement.

The moiety may be any size and have any structure. For instance, the moiety may be an oligomer, such as a dimer or trimer. The moiety is preferably a small, globular polypeptide formed from one monomer. Such moieties are easy to handle and are less likely to interfere with the ability of the modified helicase to control the movement of the polynucleotide, particularly if fused to or inserted into the sequence of the modified helicase.

The amino and carboxy terminii of the moiety are preferably in close proximity. The amino and carboxy terminii of the moiety are more preferably presented on same face of the moiety. Such embodiments facilitate insertion of the moiety into the sequence of the modified helicase. For instance, if the amino and carboxy terminii of the moiety are in close proximity, each can be attached by genetic fusion to adjacent amino acids in the sequence of the modified helicase.

It is also preferred that the location and function of the active site of the moiety is known. This prevents modifications being made to the active site that abolish the activity of the moiety. It also allows the moiety to be attached to the modified helicase so that the moiety binds to the polynucleotide and controls its movement. Knowledge of the way in which a moiety may bind to and orient polynucleotides also allows an effective construct to be designed.

The constructs of the invention are useful in Strand Sequencing. The moiety preferably binds the polynucleotide in a buffer background which is compatible with Strand Sequencing and the discrimination of the nucleotides. The moiety preferably has at least residual activity in a salt concentration well above the normal physiological level, such as from 100 mM to 2M. The moiety is more preferably modified to increase its activity at high salt concentrations. The moiety may also be modified to improve its processivity, stability and shelf life.

Suitable modifications can be determined from the characterisation of polynucleotide binding moieties from extremophiles such as halophilic, moderately halophilic bacteria, thermophilic and moderately thermophilic organisms, as well as directed evolution approaches to altering the salt tolerance, stability and temperature dependence of mesophilic or thermophilic exonucleases.

The polynucleotide binding moiety preferably comprises one or more domains independently selected from helix-hairpin-helix (HhH) domains, eukaryotic single-stranded binding proteins (SSBs), bacterial SSBs, archaeal SSBs, viral SSBs, double-stranded binding proteins, sliding clamps, processivity factors, DNA binding loops, replication initiation proteins, telomere binding proteins, repressors, zinc fingers and proliferating cell nuclear antigens (PCNAs).

The polynucleotide binding moiety may be any of those disclosed in WO 2014/013260 (incorporated herein by reference in its entirety), including Table 5 of WO 2014/013260 (incorporated herein by reference in its entirety).

The polynucleotide binding moiety is preferably derived from a polynucleotide binding enzyme. A polynucleotide binding enzyme is a polypeptide that is capable of binding to a polynucleotide and interacting with and modifying at least one property of the polynucleotide. The enzyme may modify the polynucleotide by cleaving it to form individual nucleotides or shorter chains of nucleotides, such as di- or trinucleotides. The enzyme may modify the polynucleotide by orienting it or moving it to a specific position. The polynucleotide binding moiety does not need to display enzymatic activity as long as it is capable of binding the polynucleotide and controlling its movement. For instance, the moiety may be derived from an enzyme that has been modified to remove its enzymatic activity or may be used under conditions which prevent it from acting as an enzyme.

The polynucleotide binding moiety is preferably derived from a nucleolytic enzyme. The enzyme is more preferably derived from a member of any of the Enzyme Classification (EC) groups 3.1.11, 3.1.13, 3.1.14, 3.1.15, 3.1.16, 3.1.21, 3.1.22, 3.1.25, 3.1.26, 3.1.27, 3.1.30 and 3.1.31.

The enzyme may be any of those disclosed in International Application No. PCT/GB10/000133 (published as WO 2010/086603).

Preferred enzymes are exonucleases, polymerases, helicases and topoisomerases, such as gyrases. Suitable exonucleases include, but are not limited to, exonuclease I from E. coli, exonuclease III enzyme from E. coli, RecJ from T. thermophilus and bacteriophage lambda exonuclease and variants thereof. The polynucleotide binding enzyme may be any of those disclosed in WO 2014/013260 (incorporated herein by reference in its entirety).

The moiety may be labelled with a revealing label. The label may be any of those described above.

The moiety may be isolated from any moiety-producing organism, such as E. coli, T. thermophilus or bacteriophage, or made synthetically or by recombinant means. For example, the moiety may be synthesized by in vitro translation and transcription as described below. The moiety may be produced in large scale following purification as described below.

As will be clear from the discussion above, the polynucleotide binding moiety is preferably derived from a helicase. For instance, it may be a polynucleotide domain from a helicase. The moiety more preferably comprises one or more helicases. The helicases may be any of those discussed above, including the helicases of the invention. In such embodiments, the constructs of the invention comprise two or more helicases attached together where at least one of the helicases is a helicase of the invention. All of the two or more helicases are preferably helicases of the invention. The constructs may comprise two, three, four, five or more helicases. In other words, the constructs of the invention may comprise a helicase dimer, a helicase trimer, a helicase tetramer, a helicase pentamer and the like.

The two or more helicases can be attached together in any orientation. Identical or similar helicases may be attached via the same amino acid position or spatially proximate amino acid positions in each helicase. This is termed the “head-to-head” formation. Alternatively, identical or similar helicases may be attached via positions on opposite or different sides of each helicase. This is termed the “head-to-tail” formation. Helicase trimers comprising three identical or similar helicases may comprise both the head-to-head and head-to-tail formations.

The two or more helicases may be different from one another (i.e. the construct is a hetero-dimer, -trimer, -tetramer or -pentamer etc.). The construct may comprise two different variants of the same helicase. Such constructs can be formed as described in and include any of the modified helicases disclosed in WO 2014/013260 (incorporated herein by reference in its entirety).

The two or more helicases in the constructs of the invention may be the same as one another (i.e. the construct is a homo-dimer, -trimer, -tetramer or -pentamer etc.). Homo-oligomers can comprise two or more modified helicases of the invention. Such constructs can be formed and include any of the modified helicases disclosed in WO 2014/013260 (incorporated herein by reference in its entirety).

### Method for Making Proteins

Methods for producing proteins and introducing or substituting non-naturally occurring amino acids in proteins are well known in the art. The proteins may be modified to assist their identification or purification, for example by the addition of a streptavidin tag or by the addition of a signal sequence to promote their secretion from a cell where the protein does not naturally contain such a sequence. The proteins may also be produced using D-amino acids or a mixture of L-amino acids and D-amino acids. This is conventional in the art for producing such proteins or peptides.

The protein may be chemically modified. The protein can be chemically modified in any way and at any site. The protein may be chemically modified by attachment of a molecule to one or more cysteines (cysteine linkage), attachment of a molecule to one or more lysines, attachment of a molecule to one or more non-natural amino acids, enzyme modification of an epitope or modification of a terminus. Suitable methods for carrying out such modifications are well-known in the art. The protein may be chemically modified by the attachment of any molecule, such as a dye or a fluorophore.

Any of the proteins may be modified to assist their identification or purification, for example by the addition of histidine residues (a his tag), aspartic acid residues (an asp tag), a streptavidin tag, a flag tag, a SUMO tag, a GST tag or a MBP tag, or by the addition of a signal sequence to promote their secretion from a cell where the polypeptide does not naturally contain such a sequence. An alternative to introducing a genetic tag is to chemically react a tag onto a native or engineered position on the protein. An example of this would be to react a gel-shift reagent to a cysteine engineered on the outside of the protein. This has been demonstrated as a method for separating hemolysin hetero-oligomers (Chem Biol. 1997 July; 4(7):497-505).

Any of the proteins may be labelled with a revealing label. The revealing label may be any suitable label which allows the protein to be detected. Suitable labels include, but are not limited to, fluorescent molecules, radioisotopes, e.g., 125I, 35S, enzymes, antibodies, antigens, polynucleotides, and ligands such as biotin.

The protein may also contain other non-specific modifications as long as they do not interfere with the function of the protein. A number of non-specific side chain modifications are known in the art and may be made to the side chains of the protein(s). Such modifications include, for example, reductive alkylation of amino acids by reaction with an aldehyde followed by reduction with NaBH4, amidation with methylacetimidate or acylation with acetic anhydride.

Any of the proteins can be produced using standard methods known in the art. Polynucleotide sequences encoding a protein may be derived and replicated using standard methods in the art. Polynucleotide sequences encoding a protein may be expressed in a bacterial host cell using standard techniques in the art. The protein may be produced in a cell by in situ expression of the polypeptide from a recombinant expression vector. The expression vector optionally carries an inducible promoter to control the expression of the polypeptide. These methods are described in Sambrook, J. and Russell, D. (2001). Molecular Cloning: A Laboratory Manual, 3rd Edition. Cold Spring Harbor Laboratory Press, Cold Spring Harbor, NY.

Proteins may be produced in large scale following purification by any protein liquid chromatography system from protein producing organisms or after recombinant expression. Typical protein liquid chromatography systems include FPLC, AKTA systems, the Bio-Cad system, the Bio-Rad BioLogic system, and the Gilson HPLC system.

### Method of Producing Modified Helicases and Constructs

The invention also provides a method of producing a modified NS3 of the invention. The method comprising (a) providing a NS3 helicase and (b) modifying the NS3 helicase with one or more attached linkers or a linkage to produce a modified helicase of the invention.

The one or more attached linkers or a linkage preferably form a covalently closed structure around the polynucleotide binding domain of the NS3 helicase. The one or more attached linkers or a linkage preferably modify the NS3 helicase such that it is capable of forming a covalently closed structure around the polynucleotide. The one or more attached linkers or a linkage preferably artificially covalently connect the two amino acid residues. The one or more attached linkers or a linkage preferably covalently connect the two or more parts of the helicase.

The one or more attached linkers or a linkage are preferably greater than about 8.0 angstroms (Å) in length. They may be any of the length described above with reference to the modified NS3 helicases of the invention.

The method preferably further comprises determining whether or not the modified helicase is capable of controlling the movement of a polynucleotide. Assays for doing this are described above. If the movement of a polynucleotide can be controlled, the modified helicase has been modified correctly and a modified helicase of the invention has been produced.

The invention also provides a method of producing a construct of the invention. The method comprises attaching a modified helicase of the invention to an additional polynucleotide binding moiety and thereby producing the construct. Any of the embodiments discussed above with reference to the constructs of the invention, especially in relation to WO 2014/013260 (incorporated herein by reference in its entirety), equally apply here.

The method preferably further comprises determining whether or not the construct is capable of controlling the movement of a polynucleotide. Assays for doing this are described above. If the movement of a polynucleotide can be controlled, the modified helicase and moiety have been attached correctly and a construct of the invention has been produced.

### Methods of Controlling the Movement of a Target Analyte

The invention also provides a method of controlling the movement of a target analyte. The method comprises contacting the target analyte with a modified helicase of the invention or a construct of the invention and thereby controlling the movement of the polynucleotide. The method is preferably for controlling the movement of a target analyte with respect to or through a transmembrane pore The method is preferably carried out with a potential applied across the pore. As discussed in more detail below, the applied potential typically results in the formation of a complex between the pore and the modified helicase or construct. The applied potential may be a voltage potential. Alternatively, the applied potential may be a chemical potential. An example of this is using a salt gradient across an amphiphilic layer. A salt gradient is disclosed in Holden et al., J Am Chem Soc. 2007 Jul. 11; 129(27):8650-5. Target analytes and pores are discussed in more detail below.

### Methods of Characterising a Target Analyte

The invention also provides a method of determining the presence, absence or one or more characteristics of a target analyte. The method involves contacting the target analyte with a pore or a transmembrane pore and a modified helicase of the invention or a construct of the invention such that the modified helicase or construct controls the movement of the target analyte through the pore. The method also involves taking one or more measurements as the target analyte moves with respect to the pore wherein the measurements are indicative of one or more characteristics of the target analyte and thereby determining the presence, absence or one or more characteristics of the target analyte.

The target analyte may also be called the template analyte or the analyte of interest. The modified helicase of the invention or the constrict of the invention may be any of those discussed above.

The method is for determining the presence, absence or one or more characteristics of a target analyte. The method may be for determining the presence, absence or one or more characteristics of at least one target analyte. The method may concern determining the presence, absence or one or more characteristics of two or more target analytes. The method may comprise determining the presence, absence or one or more characteristics of any number of target analytes, such as 2, 5, 10, 15, 20, 30, 40, 50, 100 or more analytes. Any number of characteristics of the one or more target analytes may be determined, such as 1, 2, 3, 4, 5, 10 or more characteristics.

The binding of a molecule in the channel of pore, or in the vicinity of either opening of the channel will have an effect on the open-channel ion flow through the pore, which is the essence of “molecular sensing”. In a similar manner to the nucleic acid sequencing application, variation in the open-channel ion flow can be measured using suitable measurement techniques by the change in electrical current (for example, WO 2000/28312 and D. Stoddart et al., Proc. Natl. Acad. Sci., 2010, 106, 7702-7 or WO 2009/077734; all incorporated herein by reference in their entirety). The degree of reduction in ion flow, as measured by the reduction in electrical current, is related to the size of the obstruction within, or in the vicinity of, the pore. Binding of a molecule of interest, also referred to as an “analyte”, in or near the pore therefore provides a detectable and measurable event, thereby forming the basis of a “biological sensor”. Suitable molecules for pore sensing include nucleic acids; proteins; peptides; polysaccharides and small molecules (refers here to a low molecular weight (e.g., <900 Da or <500 Da) organic or inorganic compound) such as pharmaceuticals, toxins, cytokines, and pollutants. Detecting the presence of biological molecules finds application in personalised drug development, medicine, diagnostics, life science research, environmental monitoring and in the security and/or the defence industry.

The pore may serve as a molecular or biological sensor. The target analyte molecule that is to be detected may bind to either face of the channel, or within the lumen of the channel itself. The position of binding may be determined by the size of the molecule to be sensed.

The target analyte preferably comprises or consists of a metal ion, an inorganic salt, a polymer, an amino acid, a peptide, a polypeptide, a protein, a nucleotide, an oligonucleotide, a polynucleotide, a polynucleotide-polypeptide conjugate, a monosaccharide, an oligosaccharide, a polysaccharide, a dye, a bleach, a pharmaceutical, a diagnostic agent, a recreational drug, an explosive, a toxic compound, or an environmental pollutant. The target analyte preferably comprises or consists of a polypeptide, a protein, an oligonucleotide, a polynucleotide, a polynucleotide-polypeptide conjugate, an oligosaccharide, or a polysaccharide. The target analyte may comprise two or more different molecules, such as a peptide and a polypeptide. The target analyte may be a polynucleotide-polypeptide conjugate. The method may concern determining the presence, absence or one or more characteristics of two or more target analytes of the same type, such as two or more proteins, two or more nucleotides or two or more pharmaceuticals. Alternatively, the method may concern determining the presence, absence or one or more characteristics of two or more target analytes of different types, such as one or more proteins, one or more nucleotides and one or more pharmaceuticals.

The target analyte can be secreted from cells. Alternatively, the target analyte can be an analyte that is present inside cells such that the target analyte must be extracted from the cells before the method can be carried out. The target analyte may be obtained from or extracted from any organism or microorganism. The target analyte may be obtained from a human or animal, e.g., from urine, lymph, saliva, mucus, seminal fluid, or amniotic fluid, or from whole blood, plasma, or serum. The target analyte may be obtained from a plant e.g., a cereal, legume, fruit, or vegetable.

The pore may be modified via recombinant or chemical methods to increase the strength of binding, the position of binding, or the specificity of binding of the molecule to be sensed. Typical modifications include addition of a specific binding moiety complimentary to the structure of the molecule to be sensed. Where the analyte molecule comprises a nucleic acid, this binding moiety may comprise a cyclodextrin or an oligonucleotide; for small molecules this may be a known complimentary binding region, for example the antigen binding portion of an antibody or of a non-antibody molecule, including a single chain variable fragment (scFv) region or an antigen recognition domain from a T-cell receptor (TCR); or for proteins, it may be a known ligand of the target protein. In this way the pore may be rendered capable of acting as a molecular sensor for detecting presence in a sample of suitable antigens (including epitopes) that may include cell surface antigens, including receptors, markers of solid tumours or haematologic cancer cells (e.g. lymphoma or leukaemia), viral antigens, bacterial antigens, protozoal antigens, allergens, allergy related molecules, albumin (e.g. human, rodent, or bovine), fluorescent molecules (including fluorescein), blood group antigens, small molecules, drugs, enzymes, catalytic sites of enzymes or enzyme substrates, and transition state analogues of enzyme substrates. As described above, modifications may be achieved using known genetic engineering and recombinant DNA techniques. The positioning of any adaptation would be dependent on the nature of the molecule to be sensed, for example, the size, three-dimensional structure, and its biochemical nature. The choice of adapted structure may make use of computational structural design. Determination and optimization of protein-protein interactions or protein-small molecule interactions can be investigated using technologies such as a BIAcore® which detects molecular interactions using surface plasmon resonance (BIAcore, Inc., Piscataway, NJ; see also www.biacore.com).

The target analyte preferably comprises or consists of an amino acid, a peptide, a polypeptides, or protein. The amino acid, peptide, polypeptide, or protein can be naturally occurring or non-naturally occurring. The polypeptide or protein can include within them synthetic or modified amino acids. Several different types of modification to amino acids are known in the art. Suitable amino acids and modifications thereof are above. It is to be understood that the target analyte can be modified by any method available in the art.

The target analyte preferably comprises a polypeptide. The term polypeptide is interchangeable with protein. Any suitable polypeptide can be characterised. The polypeptide may be an unmodified protein or a portion thereof, or a naturally occurring polypeptide or a portion thereof. The target polypeptide may be secreted from cells. Alternatively, the target polypeptide can be produced inside cells such that it must be extracted from cells for characterisation. The polypeptide may comprise the products of cellular expression of a plasmid, e.g., a plasmid used in cloning of proteins in accordance with the methods described in Sambrook et al., Molecular Cloning: A Laboratory Manual, 4^{th} ed., Cold Spring Harbor Press, Plainsview, New York (2012); and Ausubel et al., Current Protocols in Molecular Biology (Supplement 114), John Wiley & Sons, New York (2016).

The polypeptide can be provided as an impure mixture of one or more polypeptides and one or more impurities. Impurities may comprise truncated forms of the target polypeptide which are distinct from the “target polypeptides” for characterisation. For example, the target polypeptide may be a full-length protein and impurities may comprise fractions of the protein. Impurities may also comprise proteins other than the target protein, e.g., which may be co-purified from a cell culture or obtained from a sample.

A polypeptide may comprise any combination of any amino acids, amino acid analogs and modified amino acids (i.e., amino acid derivatives). Amino acids (and derivatives, analogs etc) in the polypeptide can be distinguished by their physical size and charge. The amino acids/derivatives/analogs can be naturally occurring or artificial. The polypeptide may comprise any naturally occurring amino acid.

The polypeptide may be modified. The polypeptide may be modified for detection using the method of the invention. The method may be for characterising modifications in the target polypeptide.

One or more of the amino acids/derivatives/analogs in the polypeptide may be modified. One or more of the amino acids/derivatives/analogs in the polypeptide may be post-translationally modified. As such, the method of the invention can be used to detect the presence, absence, number of positions of post-translational modifications in a polypeptide. The method can be used to characterise the extent to which a polypeptide has been post-translationally modified. Any one or more post-translational modifications may be present in the polypeptide. Typical post-translational modifications include modification with a hydrophobic group, modification with a cofactor, addition of a chemical group, glycation (the non-enzymatic attachment of a sugar), phosphorylation, biotinylation and pegylation. Post-translational modifications can also be non-natural, such that they are chemical modifications done in the laboratory for biotechnological or biomedical purposes. This can allow monitoring the levels of the laboratory made peptide, polypeptide, or protein in contrast to the natural counterparts.

Examples of post-translational modification with a hydrophobic group include myristoylation, attachment of myristate, a C₁₄ saturated acid; palmitoylation, attachment of palmitate, a C₁₆ saturated acid; isoprenylation or prenylation, the attachment of an isoprenoid group; farnesylation, the attachment of a farnesol group; geranylgeranylation, the attachment of a geranylgeraniol group; and glypiation, and glycosylphosphatidylinositol (GPI) anchor formation via an amide bond.

Examples of post-translational modification with a cofactor include lipoylation, attachment of a lipoate (C₈) functional group; flavination, attachment of a flavin moiety (e.g. flavin mononucleotide (FMN) or flavin adenine dinucleotide (FAD)); attachment of heme C, for instance via a thioether bond with cysteine; phosphopantetheinylation, the attachment of a 4′-phosphopantetheinyl group; and retinylidene Schiff base formation.

Examples of post-translational modification by addition of a chemical group include acylation, e.g. O-acylation (esters), N-acylation (amides) or S-acylation (thioesters); acetylation, the attachment of an acetyl group for instance to the N-terminus or to lysine; formylation; alkylation, the addition of an alkyl group, such as methyl or ethyl; methylation, the addition of a methyl group for instance to lysine or arginine; amidation; butyrylation; gamma-carboxylation; glycosylation, the enzymatic attachment of a glycosyl group for instance to arginine, asparagine, cysteine, hydroxylysine, serine, threonine, tyrosine or tryptophan; polysialylation, the attachment of polysialic acid; malonylation; hydroxylation; iodination; bromination; citrulination; nucleotide addition, the attachment of any nucleotide such as any of those discussed above, ADP ribosylation; oxidation; phosphorylation, the attachment of a phosphate group for instance to serine, threonine or tyrosine (O-linked) or histidine (N-linked); adenylylation, the attachment of an adenylyl moiety for instance to tyrosine (O-linked) or to histidine or lysine (N-linked); propionylation; pyroglutamate formation; S-glutathionylation; Sumoylation; S-nitrosylation; succinylation, the attachment of a succinyl group for instance to lysine; selenoylation, the incorporation of selenium; and ubiquitinilation, the addition of ubiquitin subunits (N-linked). The polypeptide may be labelled with a molecular label. A molecular label may be a modification to the polypeptide which promotes the detection of the polypeptide in the method of the invention. For example, the label may be a modification to the polypeptide which alters the signal obtained as conjugate is characterised. For example, the label may interfere with a flux of ions through the pore. In such a manner, the label may improve the sensitivity of the method. The polypeptide may contain one or more cross-linked sections, e.g., C—C bridges. The polypeptide may not be cross-linked prior to being characterised using the method.

The polypeptide may comprise sulphide-containing amino acids and thus has the potential to form disulphide bonds. Typically, in such embodiments, the polypeptide is reduced using a reagent such as DTT (Dithiothreitol) or TCEP (tris(2-carboxyethyl)phosphine) prior to being characterised using the method.

The polypeptide may be a full-length protein or naturally occurring polypeptide. The protein or naturally occurring polypeptide may be fragmented prior to conjugation to the polynucleotide. The polypeptide may be chemically or enzymatically fragmented. The polypeptides or polypeptide fragments can be conjugated to form a longer target polypeptide.

The polypeptide can be any suitable length. The polypeptide preferably has a length of from about 2 to about 300 peptide units or amino acids. The polypeptide has a length of from about 2 to about 100 peptide units, for example from about 2 to about 50 peptide units, e.g., from about 3 to about 50 peptide units, such as from about 5 to about 25 peptide units, e.g., from about 7 to about 16 peptide units, such as from about 9 to about 12 peptide units. “Peptide unit” is interchangeable with “amino acid”.

The one or more characteristics of the polypeptide are preferably selected from (i) the length of the polypeptide, (ii) the identity of the polypeptide, (iii) the sequence of the polypeptide, (iv) the secondary structure of the polypeptide and (v) whether or not the polypeptide is modified. The one or more characteristics may be the sequence of the polypeptide or whether or not the polypeptide is modified, e.g., by one or more post-translational modifications. The one or more characteristics are preferably the sequence of the polypeptide.

The polypeptide may be in a relaxed form. The polypeptide may be held in a linearized form. Holding the polypeptide in a linearized form can facilitate the characterisation of the polypeptide on a residue-by-residue basis as “bunching up” of the polypeptide within the pore is prevented. The polypeptide can be held in a linearized form using any suitable means. For example, if the polypeptide is charged, the polypeptide can be held in a linearized form by applying a voltage.

If the polypeptide is not charged or is only weakly charged then the charge can be altered or controlled by adjusting the pH. For example, the polypeptide can be held in a linearized form by using high pH to increase the relative negative charge of the polypeptide. Increasing the negative charge of the polypeptide allows it to be held in a linearized form under, e.g., a positive voltage. Alternatively, the polypeptide can be held in a linearized form by using low pH to increase the relative positive charge of the polypeptide. Increasing the positive charge of the polypeptide allows it to be held in a linearized form under, e.g., a negative voltage. In the disclosed methods a polynucleotide-handling protein is used to control the movement of a polynucleotide with respect to a pore. As a polynucleotide is typically negatively charged it is generally most suitable to increase the linearization of the polypeptide by increasing the pH thus making the polypeptide more negatively charged, in common with the polynucleotide. In this way, the conjugate retains an overall negative charge and thus can readily move, e.g., under an applied voltage.

The polypeptide can be held in a linearized form by using suitable denaturing conditions. Suitable denaturing conditions include, for example, the presence of appropriate concentrations of denaturants such as guanidine HCl and/or urea. The concentration of such denaturants to use in the disclosed methods is dependent on the target polypeptide to be characterised in the methods and can be readily selected by those of skill in the art.

The polypeptide can be held in a linearized form by using suitable detergents. Suitable detergents for use in the disclosed methods include SDS (sodium dodecyl sulfate). The polypeptide can be held in a linearized form by carrying out the disclosed methods at an elevated temperature. Increasing the temperature overcomes intra-strand bonding and allows the polypeptide to adopt a linearized form.

The polypeptide can be held in a linearized form by carrying out the method under strong electro-osmotic forces. Such forces can be provided by using asymmetric salt conditions and/or providing suitable charge in the channel of the pore. The charge in the channel of a pore can be altered, e.g., by mutagenesis. Altering the charge of a pore is well within the capacity of those skilled in the art. Altering the charge of a pore generates strong electro-osmotic forces from the unbalanced flow of cations and anions through the pore when a voltage potential is applied across the pore.

The polypeptide can be held in a linearized form by passing it through a structure such an array of nanopillars, through a nanoslit or across a nanogap. The physical constraints of such structures can force the polypeptide to adopt a linearized form.

The target analyte is preferably a polynucleotide, such as a nucleic acid, which is defined as a macromolecule comprising two or more nucleotides. Nucleic acids are particularly suitable for pore sequencing. The naturally occurring nucleic acid bases in DNA and RNA may be distinguished by their physical size. As a nucleic acid molecule, or individual base, passes through the channel of a pore, the size differential between the bases causes a directly correlated reduction in the ion flow through the channel. The variation in ion flow may be recorded. Suitable electrical measurement techniques for recording ion flow variations are discussed above. Through suitable calibration, the characteristic reduction in ion flow can be used to identify the particular nucleotide and associated base traversing the channel in real-time. In typical pore nucleic acid sequencing, the open-channel ion flow is reduced as the individual nucleotides of the nucleic sequence of interest sequentially pass through the channel of the pore due to the partial blockage of the channel by the nucleotide. It is this reduction in ion flow that is measured using the suitable recording techniques described above. The reduction in ion flow may be calibrated to the reduction in measured ion flow for known nucleotides through the channel resulting in a means for determining which nucleotide is passing through the channel, and therefore, when done sequentially, a way of determining the nucleotide sequence of the nucleic acid passing through the pore. For the accurate determination of individual nucleotides, it has typically required for the reduction in ion flow through the channel to be directly correlated to the size of the individual nucleotide passing through the constriction. It will be appreciated that sequencing may be performed upon an intact nucleic acid polymer that is ‘threaded’ through the pore via the action of an associated polymerase, for example. Alternatively, sequences may be determined by passage of nucleotide triphosphate bases that have been sequentially removed from a target nucleic acid in proximity to the pore (see for example WO 2014/187924 incorporated herein by reference in its entirety).

The polynucleotide or nucleic acid may comprise any combination of any nucleotides. The nucleotides can be naturally occurring or artificial. One or more nucleotides in the polynucleotide can be oxidized or methylated. One or more nucleotides in the polynucleotide may be damaged. For instance, the polynucleotide may comprise a pyrimidine dimer. Such dimers are typically associated with damage by ultraviolet light and are the primary cause of skin melanomas. One or more nucleotides in the polynucleotide may be modified, for instance with a label or a tag, for which suitable examples are known by a skilled person. The polynucleotide may comprise one or more spacers. A nucleotide typically contains a nucleobase, a sugar and at least one phosphate group. The nucleobase and sugar form a nucleoside. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar is preferably a deoxyribose. The polynucleotide preferably comprises the following nucleosides: deoxyadenosine (dA), deoxyuridine (dU) and/or thymidine (dT), deoxyguanosine (dG) and deoxycytidine (dC). The nucleotide is typically a ribonucleotide or deoxyribonucleotide. The nucleotide typically contains a monophosphate, diphosphate, or triphosphate. The nucleotide may comprise more than three phosphates, such as 4 or 5 phosphates. Phosphates may be attached on the 5′ or 3′ side of a nucleotide. The nucleotides in the polynucleotide may be attached to each other in any manner. The nucleotides are typically attached by their sugar and phosphate groups as in nucleic acids. The nucleotides may be connected via their nucleobases as in pyrimidine dimers. The polynucleotide may be single stranded or double stranded. At least a portion of the polynucleotide is preferably double stranded. The polynucleotide is most preferably ribonucleic nucleic acid (RNA) or deoxyribonucleic acid (DNA). In particular, said method using a polynucleotide as an analyte alternatively comprises determining one or more characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified.

The polynucleotide can be any length (i). For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length. Any number of polynucleotides can be investigated. For instance, the method may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide. The polynucleotide can be naturally occurring or artificial. For instance, the method may be used to verify the sequence of a manufactured oligonucleotide. The method is typically carried out in vitro.

Nucleotides can have any identity (ii), and include, but are not limited to, adenosine monophosphate (AMP), guanosine monophosphate (GMP), thymidine monophosphate (TMP), uridine monophosphate (UMP), 5-methylcytidine monophosphate, 5-hydroxymethylcytidine monophosphate, cytidine monophosphate (CMP), cyclic adenosine monophosphate (cAMP), cyclic guanosine monophosphate (cGMP), deoxyadenosine monophosphate (dAMP), deoxyguanosine monophosphate (dGMP), deoxythymidine monophosphate (dTMP), deoxyuridine monophosphate (dUMP), deoxycytidine monophosphate (dCMP) and deoxymethylcytidine monophosphate. The nucleotides are preferably selected from AMP, TMP, GMP, CMP, UMP, dAMP, dTMP, dGMP, dCMP and dUMP. A nucleotide may be abasic (i.e., lack a nucleobase). A nucleotide may also lack a nucleobase and a sugar (i.e., is a C3 spacer). The sequence of the nucleotides (iii) is determined by the consecutive identity of following nucleotides attached to each other throughout the polynucleotide strain, in the 5′ to 3′ direction of the strand.

In particular, said method using a polynucleotide as an analyte alternatively comprises determining one or more characteristics selected from (i) the length of the polynucleotide, (ii) the identity of the polynucleotide, (iii) the sequence of the polynucleotide, (iv) the secondary structure of the polynucleotide and (v) whether or not the polynucleotide is modified.

The polynucleotide can be any length (i). For example, the polynucleotide can be at least 10, at least 50, at least 100, at least 150, at least 200, at least 250, at least 300, at least 400 or at least 500 nucleotides or nucleotide pairs in length. The polynucleotide can be 1000 or more nucleotides or nucleotide pairs, 5000 or more nucleotides or nucleotide pairs in length or 100000 or more nucleotides or nucleotide pairs in length. Any number of polynucleotides can be investigated. For instance, the method may concern characterising 2, 3, 4, 5, 6, 7, 8, 9, 10, 20, 30, 50, 100 or more polynucleotides. If two or more polynucleotides are characterised, they may be different polynucleotides or two instances of the same polynucleotide. The polynucleotide can be naturally occurring or artificial. For instance, the method may be used to verify the sequence of a manufactured oligonucleotide. The method is typically carried out in vitro.

Nucleotides can have any identity (ii). Possible nucleotides are defined above. The sequence of the nucleotides (iii) is determined by the consecutive identity of following nucleotides attached to each other throughout the polynucleotide strain, in the 5′ to 3′ direction of the strand.

The method is particularly useful in analysing homopolymers. For example, they may be used to determine the sequence of a polynucleotide comprising two or more, such as at least 3, 4, 5, 6, 7, 8, 9 or 10, consecutive nucleotides that are identical. For example, they may be used to sequence a polynucleotide comprising a polyA, polyT, polyG and/or polyC region.

The target analyte may comprise a polynucleotide and a polypeptide. The target analyte may be a polynucleotide-polypeptide conjugate. The conjugate preferably comprises a polynucleotide conjugated to a polypeptide. One or both of the polynucleotide and polypeptide may be the target and may be characterised in accordance with the invention.

The polypeptide can be conjugate to the polynucleotide at any suitable position. For example, the polypeptide can be conjugated to the polynucleotide at the N-terminus or the C-terminus of the polypeptide. The polypeptide can be conjugated to the polynucleotide via a side chain group of a residue (e.g., an amino acid residue) in the polypeptide. The polypeptide may have a naturally occurring reactive functional group which can be used to facilitate conjugation to the polynucleotide. For example, a cysteine residue can be used to form a disulphide bond to the polynucleotide or to a modified group thereon.

The polypeptide may be modified in order to facilitate its conjugation to the polynucleotide. For example, the polypeptide may be modified by attaching a moiety comprising a reactive functional group for attaching to the polynucleotide. For example, the polypeptide can be extended at the N-terminus or the C-terminus by one or more residues (e.g., amino acid residues) comprising one or more reactive functional groups for reacting with a corresponding reactive functional group on the polynucleotide. For example, the polypeptide can be extended at the N-terminus and/or the C-terminus by one or more cysteine residues. Such residues can be used for attachment to the polynucleotide portion of the conjugate, e.g., by maleimide chemistry (e.g., by reaction of cysteine with an azido-maleimide compound such as azido-[Pol]-maleimide wherein [Pol] is typically a short chain polymer such as PEG, e.g., PEG2, PEG3, or PEG4; followed by coupling to appropriately functionalised polynucleotide e.g., polynucleotide carrying a BCN group for reaction with the azide). Such chemistry is described in Example 2. For avoidance of doubt, when the polypeptide comprises an appropriate naturally occurring residue at the N- and/or C-terminus (e.g., a naturally occurring cysteine residue at the N- and/or C-terminus) then such residue(s) can be used for attachment to the polynucleotide.

A residue in the polypeptide may be modified to facilitate attachment of the polypeptide to the polynucleotide. A residue (e.g., an amino acid residue) in the polypeptide may be chemically modified for attachment to the polynucleotide. A residue (e.g., an amino acid residue) in the polypeptide may be enzymatically modified for attachment to the polynucleotide.

The conjugation chemistry between the polynucleotide and the polypeptide in the conjugate is not particularly limited. Any suitable combination of reactive functional groups can be used. Many suitable reactive groups and their chemical targets are known in the art. Some exemplary reactive groups and their corresponding targets include aryl azides which may react with amine, carbodiimides which may react with amines and carboxyl groups, hydrazides which may react with carbohydrates, hydroxmethyl phosphines which may react with amines, imidoesters which may react with amines, isocyanates which may react with hydroxyl groups, carbonyls which may react with hydrazines, maleimides which may react with sulfhydryl groups, NHS-esters which may react with amines, PFP-esters which may react with amines, psoralens which may react with thymine, pyridyl disulfides which may react with sulfhydryl groups, vinyl sulfones which may react with sulfhydryl amines and hydroxyl groups, vinylsulfonamides, and the like. Other suitable chemistry for conjugating the polypeptide to the polynucleotide includes click chemistry. Many suitable click chemistry reagents are known in the art. Suitable examples of click chemistry include, but are not limited to, the following:
copper (I)-catalyzed azide-alkyne cycloadditions (azide alkyne Huisgen cycloadditions);
strain-promoted azide-alkyne cycloadditions; including alkene and azide [3+2] cycloadditions;
alkene and tetrazine inverse-demand Diels-Alder reactions; and alkene and tetrazole photoclick reactions;
copper-free variant of the 1,3 dipolar cycloaddition reaction, where an azide reacts with an alkyne under strain, for example in a cyclooctane ring such as in bicycle[6.1.0]nonyne (BCN);
the reaction of an oxygen nucleophile on one linker with an epoxide or aziridine reactive moiety on the other; and
the Staudinger ligation, where the alkyne moiety can be replaced by an aryl phosphine, resulting in a specific reaction with the azide to give an amide bond.

Any reactive group may be used to form the conjugate. Some suitable reactive groups include [1, 4-Bis[3-(2-pyridyldithio) propionamido]butane; 1,1 1-bis-maleimidotriethyleneglycol; 3,3′-dithiodipropionic acid di(N-hydroxysuccinimide ester); ethylene glycol-bis(succinic acid N-hydroxysuccinimide ester); 4,4′-diisothiocyanatostilbene-2,2′-disulfonic acid disodium salt; Bis[2-(4-azidosalicylamido)ethyl]disulphide; 3-(2-pyridyldithio) propionic acid N-hydroxysuccinimide ester; 4-maleimidobutyric acid N-hydroxysuccinimide ester; Iodoacetic acid N-hydroxysuccinimide ester; S-acetylthioglycolic acid N-hydroxysuccinimide ester; azide-PEG-maleimide; and alkyne-PEG-maleimide. The reactive group may be any of those disclosed in WO 2010/086602, particularly in Table 3 of that application.

The reactive functional group may be comprised in the polynucleotide and the target functional group may be comprised in the polypeptide prior to the conjugation step. The reactive functional group may be comprised in the polypeptide and the target functional group may be comprised in the polynucleotide prior to the conjugation step. The reactive functional group may be attached directly to the polypeptide. The reactive functional group may be attached to the polypeptide via a spacer. Any suitable spacer can be used. Suitable spacers include for example alkyl diamines such as ethyl diamine, etc.

The conjugate may comprise a plurality of polypeptide sections and/or a plurality of polynucleotide sections. For example, the conjugate may comprise a structure of the form . . . P—N—P—N—P—N . . . wherein P is a polypeptide and N is a polynucleotide. A polynucleotide-handling protein may sequentially control the N portions of the conjugate with respect to the pore and thus sequentially controls the movement of the P sections with respect to the pore, thus allowing the sequential characterisation of the P sections. The plurality of polynucleotides and polypeptides may be conjugated together by the same or different chemistries.

The conjugate may comprise a leader. Any suitable leader may be used. The leader may be a polynucleotide. The leader may be the same sort of polynucleotide as the polynucleotide used in the conjugate, or it may be a different type of polynucleotide. For example, the polynucleotide in the conjugate may be DNA and the leader may be RNA or vice versa.

The leader may be a charged polymer, e.g., a negatively charged polymer. The leader may comprise a polymer such as PEG or a polysaccharide. The leader may be from 10 to 150 monomer units (e.g., ethylene glycol or saccharide units) in length, such as from 20 to 120, e.g., 30 to 100, for example 40 to 80 such as 50 to 70 monomer units (e.g., ethylene glycol or saccharide units) in length. The methods of characterising a target polypeptide of the invention may comprise conjugating a polypeptide to a polynucleotide.

Any suitable pore can be used in the method of the invention. The pore is preferably a transmembrane pore. A transmembrane pore is a structure that crosses the membrane to some degree. It permits hydrated ions driven by an applied potential to flow across or within the membrane. The transmembrane pore typically crosses the entire membrane so that hydrated ions may flow from one side of the membrane to the other side of the membrane. However, the transmembrane pore does not have to cross the membrane. It may be closed at one end. For instance, the pore may be a well, gap, channel, trench or slit in the membrane along which or into which hydrated ions may flow.

The pore typically has a first opening and a second opening. The first opening is typically the cis opening and the second opening is typically the trans opening. However, the first opening may be the trans opening and the second opening may be the cis opening. The helicase or construct used in the method of the invention is typically provided at the first opening of the pore and thus controls the movement of the target polynucleotide in the direction from the second opening of the pore towards the first opening of the pore.

Any transmembrane pore may be used in the method of the invention. The pore may be biological or artificial. Suitable pores include, but are not limited to, protein pores, polynucleotide pores and solid-state pores. The pore may be a DNA origami pore (Langecker et al., Science, 2012; 338:932-936). Suitable DNA origami pores are disclosed in WO2013/083983.

The pore is preferably a transmembrane protein pore. A transmembrane protein pore is a polypeptide or a collection of polypeptides that permits hydrated ions, such as polynucleotide, to flow from one side of a membrane to the other side of the membrane. In the method of the invention, the transmembrane protein pore is capable of forming a pore that permits hydrated ions driven by an applied potential to flow from one side of the membrane to the other. The transmembrane protein pore preferably permits polynucleotides to flow from one side of the membrane, such as a triblock copolymer membrane, to the other. The transmembrane protein pore allows a polynucleotide to be moved through the pore.

The pore may be a transmembrane protein pore which is a monomer or an oligomer. The pore is preferably made up of several repeating subunits, such as at least about 6, at least about 7, at least about 8, at least about 9, at least about 10, at least about 11, at least about 12, at least about 13, at least about 14, at least about 15, or at least about 16 subunits. The pore is preferably a hexameric, heptameric, octameric or nonameric pore. The pore may be a homo-oligomer or a hetero-oligomer.

The transmembrane protein pore may comprise a barrel or channel through which the ions may flow. The subunits of the pore typically surround a central axis and contribute strands to a transmembrane β-barrel or channel or a transmembrane α-helix bundle or channel.

Typically, the barrel or channel of the transmembrane protein pore comprises amino acids that facilitate interaction with an analyte, such as a target polynucleotide (as described herein). These amino acids are preferably located near a constriction of the barrel or channel. The transmembrane protein pore typically comprises one or more positively charged amino acids, such as arginine, lysine or histidine, or aromatic amino acids, such as tyrosine or tryptophan. These amino acids typically facilitate the interaction between the pore and nucleotides, polynucleotides, or nucleic acids.

The pore may be a transmembrane protein pore derived from β-barrel pores or α-helix bundle pores. β-barrel pores comprise a barrel or channel that is formed from β-strands. Suitable β-barrel pores include, but are not limited to, β-toxins, such as α-hemolysin, anthrax toxin and leukocidins, and outer membrane proteins/porins of bacteria, such as Mycobacterium smegmatis porin (Msp), for example MspA, MspB, MspC or MspD, CsgG, outer membrane porin F (OmpF), outer membrane porin G (OmpG), outer membrane phospholipase A and Neisseria autotransporter lipoprotein (NalP) and other pores, such as lysenin. α-helix bundle pores comprise a barrel or channel that is formed from α-helices. Suitable α-helix bundle pores include, but are not limited to, inner membrane proteins and α outer membrane proteins, such as WZA and ClyA toxin.

The pore may be a transmembrane pore derived from or based on Msp, α-hemolysin (α-HL), lysenin, CsgG, ClyA, Sp1 or haemolytic protein fragaceatoxin C (FraC).

The pore may be a transmembrane protein pore derived from CsgG, e.g., from CsgG from E. coli Str. K-12 substr. MC4100. Such a pore is oligomeric and typically comprises 7, 8, 9 or 10 monomers derived from CsgG. The pore may be a homo-oligomeric pore derived from CsgG comprising identical monomers. Alternatively, the pore may be a hetero-oligomeric pore derived from CsgG comprising at least one monomer that differs from the others. Examples of suitable pores derived from CsgG are disclosed in WO 2016/034591, WO 2017/149316, WO 2017/149317, WO 2017/149318, and WO 2019/002893 (all of which are incorporated herein by reference in their entireties).

The pore may be a transmembrane pore derived from lysenin. Examples of suitable pores derived from lysenin are disclosed in WO 2013/153359 (incorporated herein by reference in its entirety).

The pore may be a transmembrane pore derived from or based on α-hemolysin (α-HL). The wild type α-hemolysin pore is formed of 7 identical monomers or sub-units (i.e., it is heptameric). An α-hemolysin pore may be α-hemolysin-NN or a variant thereof. The variant preferably comprises N residues at positions E111 and K147.

The pore may be a transmembrane protein pore derived from Msp, e.g., from MspA. Examples of suitable pores derived from MspA are disclosed in WO 2012/107778 (incorporated herein by reference in its entirety).

The pore may be a transmembrane pore derived from or based on ClyA.

The pore is typically present in a membrane. Any suitable membrane may be used. The membrane is preferably an amphiphilic layer. An amphiphilic layer is a layer formed from amphiphilic molecules, such as phospholipids, which have both hydrophilic and lipophilic properties. The amphiphilic molecules may be synthetic or naturally occurring. Non-naturally occurring amphiphiles and amphiphiles which form a monolayer are known in the art and include, for example, block copolymers (Gonzalez-Perez et al., Langmuir, 2009, 25, 10447-10450). Block copolymers are polymeric materials in which two or more monomer sub-units that are polymerized together to create a single polymer chain. Block copolymers typically have properties that are contributed by each monomer sub-unit. However, a block copolymer may have unique properties that polymers formed from the individual sub-units do not possess. Block copolymers can be engineered such that one of the monomer sub-units is hydrophobic (i.e., lipophilic), whilst the other sub-unit(s) are hydrophilic whilst in aqueous media. In this case, the block copolymer may possess amphiphilic properties and may form a structure that mimics a biological membrane. The block copolymer may be a diblock (consisting of two monomer sub-units) but may also be constructed from more than two monomer sub-units to form more complex arrangements that behave as amphipiles. The copolymer may be a triblock, tetrablock or pentablock copolymer. The membrane may be a triblock copolymer membrane.

Archaebacterial bipolar tetraether lipids are naturally occurring lipids that are constructed such that the lipid forms a monolayer membrane. These lipids are generally found in extremophiles that survive in harsh biological environments, thermophiles, halophiles and acidophiles. Their stability is believed to derive from the fused nature of the final bilayer. It is straightforward to construct block copolymer materials that mimic these biological entities by creating a triblock polymer that has the general motif hydrophilic-hydrophobic-hydrophilic. This material may form monomeric membranes that behave similarly to lipid bilayers and encompass a range of phase behaviours from vesicles through to laminar membranes. Membranes formed from these triblock copolymers hold several advantages over biological lipid membranes. Because the triblock copolymer is synthesised, the exact construction can be carefully controlled to provide the correct chain lengths and properties required to form membranes and to interact with pores and other proteins.

Block copolymers may also be constructed from sub-units that are not classed as lipid sub-materials; for example, a hydrophobic polymer may be made from siloxane or other non-hydrocarbon-based monomers. The hydrophilic sub-section of block copolymer can also possess low protein binding properties, which allows the creation of a membrane that is highly resistant when exposed to raw biological samples. This head group unit may also be derived from non-classical lipid head-groups.

Triblock copolymer membranes also have increased mechanical and environmental stability compared with biological lipid membranes, for example a much higher operational temperature or pH range. The synthetic nature of the block copolymers provides a platform to customise polymer-based membranes for a wide range of applications.

The membrane may be one of the membranes disclosed in International Application No. WO2014/064443 or WO2014/064444 (both of which are incorporated herein by reference in their entireties).

The amphiphilic molecules may be chemically modified or functionalised to facilitate coupling of the polynucleotide. The amphiphilic layer may be a monolayer or a bilayer. The amphiphilic layer is typically planar. The amphiphilic layer may be curved. The amphiphilic layer may be supported.

Amphiphilic membranes are typically naturally mobile, essentially acting as two-dimensional fluids with lipid diffusion rates of approximately 10⁻⁸ cm s⁻¹. This means that the pore and coupled polynucleotide can typically move within an amphiphilic membrane.

The membrane may be a lipid bilayer. Lipid bilayers are models of cell membranes and serve as excellent platforms for a range of experimental studies. For example, lipid bilayers can be used for in vitro investigation of membrane proteins by single-channel recording. Alternatively, lipid bilayers can be used as biosensors to detect the presence of a range of substances. The lipid bilayer may be any lipid bilayer. Suitable lipid bilayers include, but are not limited to, a planar lipid bilayer, a supported bilayer, or a liposome. The lipid bilayer is preferably a planar lipid bilayer. Suitable lipid bilayers are disclosed in WO 2008/102121, WO 2009/077734, and WO 2006/100484 (incorporated herein by reference in their entireties).

Methods for forming lipid bilayers are known in the art. Lipid bilayers are commonly formed by the method of Montal and Mueller (Proc. Natl. Acad. Sci. USA., 1972; 69:3561-3566). A lipid bilayer may be formed as described in WO 2009/077734 (incorporated herein by reference in its entirety). In this method, the lipid bilayer is formed from dried lipids. A lipid bilayer may be formed across an opening as described in WO2009/077734.

The membrane may comprise a solid-state layer. Solid state layers can be formed from both organic and inorganic materials including, but not limited to, microelectronic materials, insulating materials such as Si₃N₄, Al₂O₃, and SiO, organic and inorganic polymers such as polyamide, plastics such as Teflon® or elastomers such as two-component addition-cure silicone rubber, and glasses. The solid-state layer may be formed from graphene. Suitable graphene layers are disclosed in WO 2009/035647 (incorporated herein by reference in its entirety). If the membrane comprises a solid-state layer, the pore is typically present in an amphiphilic membrane or layer contained within the solid-state layer, for instance within a hole, well, gap, channel, trench or slit within the solid-state layer. The skilled person can prepare suitable solid state/amphiphilic hybrid systems. Suitable systems are disclosed in WO 2009/020682 and WO 2012/005857 (incorporated herein by reference in their entireties). Any of the amphiphilic membranes or layers discussed above may be used.

The methods disclosed herein are typically carried out using (i) an artificial amphiphilic layer comprising a pore, (ii) an isolated, naturally occurring lipid bilayer comprising a pore, or (iii) a cell having a pore inserted therein. The methods are typically carried out using an artificial amphiphilic layer, such as an artificial triblock copolymer layer. The layer may comprise other transmembrane and/or intramembrane proteins as well as other molecules in addition to the pore. Suitable apparatus and conditions are discussed below. The method of the invention is typically carried out in vitro.

The method for determining the presence, absence or one or more characteristics of a target polynucleotide may involve the use of one or more sequencing adaptors. The skilled person is capable of using sequencing adaptors, such as the adaptors described in WO 2016/034591 and WO 2018/100370 (both incorporated herein by reference in their entirety), to attach a suitable portion or region to a double stranded polynucleotide. These adaptors also comprise suitable binding sites for polynucleotide binding proteins, including the helicase of the invention or the construct of the invention. The skilled person is also capable of designing a functional binding moiety comprising a portion or region that is capable of hybridising to the revealed portion or region.

In any of the methods, the one or more characteristics of the target analyte are preferably measured by electrical measurement and/or optical measurement. The electrical measurement is a current measurement, an impedance measurement, a tunnelling measurement, or a field effect transistor (FET) measurement. The method preferably comprises measuring the current flowing through the pore as the target analyte moves with respect to, such as through, the pore. General conditions for conducting the methods of the invention are discussed in more detail below.

### Other Methods

The invention also provides a method of forming a sensor for characterising a target analyte, preferably a target polynucleotide. The method comprises forming a complex between a pore and a helicase of the invention or a construct of the invention. The complex may be formed by contacting the pore and the helicase or construct in the presence of the target analyte and then applying a potential across the pore. The applied potential may be a chemical potential or a voltage potential as described above. Alternatively, the complex may be formed by covalently attaching the pore to the helicase or construct. Methods for covalent attachment are known in the art and disclosed, for example, in International Application Nos. PCT/GB09/001679 (published as WO 2010/004265) and PCT/GB10/000133 (published as WO 2010/086603). The complex is a sensor for characterising the target analyte, preferably the target polynucleotide. Any of the embodiments discussed above with reference to the methods of the invention equally apply to this method. The invention also provides a sensor produced using the method of the invention. Any of the embodiments discussed above with reference to the methods of the invention equally apply to this sensor.

### Kits

The invention also provides kits for characterising a target analyte, preferably a target polynucleotide. The kit comprises a helicase of the invention or a kit of the invention. The kit also comprises one or more loading moieties. The loading moieties allow the helicase or the construct to be loaded onto the target analyte such that the helicase or construct can control the movement of the target analyte, for instance through a pore. The one or more loading moieties are preferably one or more sequencing adaptors. The skilled person is capable of using sequencing adaptors, such as the adaptors described in WO 2016/034591 and WO 2018/100370 (both incorporated herein by reference in their entirety), to attach a suitable portion or region to a double stranded polynucleotide. These adaptors also comprise suitable binding sites for polynucleotide binding proteins, including the helicase of the invention or the construct of the invention. The skilled person is also capable of designing a functional binding moiety comprising a portion or region that is capable of hybridising to the revealed portion or region.

The kit may further comprise one or more anchors, such as cholesterol, for coupling the target analyte to a membrane. The anchor, such as cholesterol, is preferably attached to the one or more loading moieties or one or more sequencing adaptors.

The kit may comprise components of any type of membranes, such as an amphiphilic layer, such as a triblock copolymer membrane. The membrane is preferably artificial. Suitable membranes are described above.

The kit may comprise a pore. The pore may be any of the pores described above. The pore is preferably within a membrane.

The kit may additionally comprise one or more other reagents or instruments which enable any of the embodiments mentioned above to be carried out. Such reagents or instruments include one or more of the following: suitable buffer(s) (aqueous solutions), means to obtain a sample from a subject (such as a vessel or an instrument comprising a needle), means to amplify and/or express polynucleotides or voltage or patch clamp apparatus. Reagents may be present in the kit in a dry state such that a fluid sample resuspends the reagents. The kit may also, optionally, comprise instructions to enable the kit to be used in the method of the invention or details regarding for which organism the method may be used. Finally, the kit may also comprise additional components useful in analyte characterization.

### Apparatus

The invention also provides an apparatus for characterising target analytes in a sample, comprising (a) a plurality of pores and (b) a plurality of helicases of the invention or a plurality of constructs of the invention. The plurality of pores may be any of those discussed above.

Any of the specific embodiments discussed above, especially in relation to the pores, helicases, and constructs, are equally applicable to the apparatuses of the invention.

The apparatus may be any of those described in WO 2009/077734, WO 2010/122293, WO 2011/067559 and WO 00/28312 (all incorporated herein by reference in their entireties).

### System

The invention provides a system comprising (a) a membrane comprising a pore, (c) a helicase of the invention or a construct of the invention, (b) means for applying a potential across the membrane(s) and (c) means for detecting electrical or optical signals across the membrane(s).

The electrical signal may be a measurement of ion flow through the pore such as the measurement of a current or voltage over time.

The membrane and pores may be any of those described above. The modified helicase of the invention or the construct of the invention may be any of those described above.

In one embodiment, the system further comprises a first chamber and a second chamber, wherein the first and second chambers are separated by the membrane(s). When used to characterise a target analyte, the system may further comprise a target analyte, wherein the target analyte is transiently located within the continuous channel and wherein one end of the target analyte is located in the first chamber and one end of the target analyte is located in the second chamber. The target analyte is preferably a target polynucleotide.

In one embodiment, the system further comprises an electrically conductive solution in contact with the pore(s), electrodes providing a voltage potential across the membrane(s), and a measurement system for measuring the current through the pore(s). The voltage applied across the membranes and pore is preferably from +5 V to −5 V, such as −600 mV to +600 mV or −400 mV to +400 mV. The voltage used is preferably in the range 100 mV to 240 mV and more preferably in the range of 120 mV to 220 mV. It is possible to increase discrimination between different amino acids or nucleotides by a pore by using an increased applied potential. Any suitable electrically conductive solution may be used. For example, the solution may comprise charge carriers, such as metal salts, for example alkali metal salt, halide salts, for example chloride salts, such as alkali metal chloride salt. Charge carriers may include ionic liquids or organic salts, for example tetramethyl ammonium chloride, trimethylphenyl ammonium chloride, phenyltrimethyl ammonium chloride, or 1-ethyl-3-methyl imidazolium chloride. In an exemplary system, salt is present in the aqueous solution in the chamber. Potassium chloride (KCl), sodium chloride (NaCl), caesium chloride (CsCl) or a mixture of potassium ferrocyanide and potassium ferricyanide is typically used. KCl, NaCl and a mixture of potassium ferrocyanide and potassium ferricyanide are preferred. The charge carriers may be asymmetric across the membrane. For instance, the type and/or concentration of the charge carriers may be different on each side of the membrane, e.g., in each chamber.

The salt concentration may be at saturation. The salt concentration may be 3 M or lower and is typically from 0.1 to 2.5 M, from 0.3 to 1.9 M, from 0.5 to 1.8 M, from 0.7 to 1.7 M, from 0.9 to 1.6 M or from 1 M to 1.4 M. The salt concentration is preferably from 150 mM to 1 M. The method is preferably carried out using a salt concentration of at least 0.3 M, such as at least 0.4 M, at least 0.5 M, at least 0.6 M, at least 0.8 M, at least 1.0 M, at least 1.5 M, at least 2.0 M, at least 2.5 M or at least 3.0 M. High salt concentrations provide a high signal to noise ratio and allow for currents indicative of the presence of an amino acid or nucleotide to be identified against the background of normal current fluctuations.

A buffer may be present in the electrically conductive solution. Typically, the buffer is phosphate buffer. Other suitable buffers are HEPES and Tris-HCl buffer. The pH of the electrically conductive solution may be from 4.0 to 12.0, from 4.5 to 10.0, from 5.0 to 9.0, from 5.5 to 8.8, from 6.0 to 8.7 or from 7.0 to 8.8 or 7.5 to 8.5. The pH used is preferably about 7.5.

The system may be comprised in an apparatus. The apparatus may be any conventional apparatus for analyte analysis, such as an array or a chip. The apparatus is preferably set up to carry out the disclosed method. For example, the apparatus may comprise a chamber comprising an aqueous solution and a barrier that separates the chamber into two sections. The barrier typically has an aperture in which the membrane(s) containing the pore(s) are formed. Alternatively, the barrier forms the membrane in which the pore is present.

The apparatus may also comprise an electrical circuit capable of applying a potential and measuring an electrical signal across the membrane and pore.

The apparatus may be any of those described in WO 2008/102120, WO 2009/077734, WO 2010/122293, WO 2011/067559, WO 2014/06442, or WO2020/183172 (all incorporated herein by reference in their entirety).

### Analysis of Measurements

The method for determining the presence, absence or one or more characteristics of a target polymer analyte may comprise estimating or determining the sequence of polymer units. The signal measured during movement of the polymer, such as a polypeptide, polynucleotide, or polypeptide-polynucleotide conjugate, with respect to the pore may be dependent at any one time upon multiple polymer units such as amino acids or nucleotides. For example, the presence of multiple amino acids or nucleotides in the lumen of the pore and potentially amino acids or nucleotides outside of the pore can influence the ion flow and therefore current or voltage signal. The polypeptide or polynucleotide may also contain modified amino acids or nucleotides which can affect the measurement signal and as such the estimation or determination of the sequence may be non-trivial. Various known mathematical techniques and variations thereof may be used to determine or estimate the polymer sequence, including probabilistic and machine learning techniques. Such methods are described for example in WO2013041878, WO2013121224, WO2018203084 and Zhang et al: A Guide to Signal Processing Algorithms for pore Sensors, ACS Sens. 2021, 6, 10, 3536-3555, all of which are hereby incorporated by reference in their entirety.

The method of the invention may comprise the measurement of target analyte wherein measurements can be used to estimate or determine an overall sequence. Various known methods may be used. For example, the sequence may be initially determined from the series of measurements taken during the movement of the analyte with respect to the pore and the results combined to provide an overall sequence. More preferably the series of measurements may be treated by a probabilistic or machine learning technique as plural series of measurements in plural dimensions wherein an overall sequence determination is made without the initial determination of the sequence of the analyte. A non-limiting example of a method suitable for use in the invention is disclosed in WO2015140535 (incorporated by reference in its entirety). The following Example illustrates the invention. It is to be understood that although particular embodiments, specific configurations as well as materials and/or molecules, have been discussed herein for engineered cells and methods according to the present invention, various changes or modifications in form and detail may be made without departing from the scope and spirit of this invention. The following example is provided to better illustrate particular embodiments, and they should not be considered limiting the application. The application is limited only by the claims.

### Example

This Example describes a method of comparing the speed of multiple polynucleotide binding proteins which have been closed by covalently linking two amino acids contained within those polynucleotide binding proteins with various crosslinking molecules with spacer arms of different lengths. The translocation speed of these differentially closed proteins is then compared using a nanopore.

### Analyte Preparation

A 1.3 kb single stranded RNA transcript of YHR174W (ENO2 from S. cerevisiae) was prepared from a DNA template amplicon using a MEGAscript T7 Transcription Kit (Invitrogen). The resulting RNA product was then purified using a MEGAclear Transcription Clean-Up Kit (Invitrogen) and quantified via a Nanodrop OneC spectrophotometer (Thermo Fisher Scientific). The final sample was considered pure if it had a A260/A280 ratio of 1.8-2.2.

### Sequencing Adaptor Preparation

Recombinant expression vectors encoding the variants of polynucleotide binding proteins and an affinity tag were transformed into chemically competent BL21 (DE3) E. coli cells. Following outgrowth in SOC media for one hour at 37° C., the cells were plated onto an LB agar plate containing antibiotics and cultured overnight at 37° C. Individual colonies from the agar plate were inoculated into 100 ml TB media containing 0.2% w/v glucose and antibiotics before being cultured overnight at 37° C. Following this, a 1:100 dilution of this culture was made into fresh TB media with glucose and antibiotics and allowed to grow at 37° C. until the O.D was between 0.45 and 0.5. At this point, the temperature was slowly reduced to 18° C. and the cells allowed to divide for a further hour, after which induction was achieved by adding IPTG to a final concentration of 0.4 mM and leaving overnight at 18° C. Cells was then be harvested by centrifugation at 6000 g for 30 mins and removing all liquids from the cell pellet. This cell pellet was then further purified prior to adaptor loading so that only the polynucleotide binding protein remains via affinity chromatography.

A molar excess of purified polynucleotide binding protein (see Table 1 below showing the different variants) was bound to sequencing Y adaptors in 25 mM HEPES, 50 mM potassium chloride, 10% glycerol, 5 mM EDTA (pH7) for 10 minutes at ambient temperature. To this solution, 0.5 μl of 1 M MgCl₂ was added per 100 μl of complex volume and incubated for 10 minutes at ambient temperature. TMAD was then added at a final concentration of 100 μM to close the enzyme with a disulphide bond, whilst the crosslinking molecules used (see Table 2) were added to a final concentration of 48 μM. All samples were then incubated at 37° C. for one hour.

TABLE 1
Different variants tested in this Example.

| | | Linker |
|---|---|---|
| Variant # | NS3 | attachment points |
| Variant 1 | NS3-HCV-JFH1 | G210C/G370C |
| Variant 2 | NS3-DV | I199C/M439C |
| Variant 3 | NS3-DV | I199C/P438C |

TABLE 2
Linker and catalyst molecules used to close variant
polynucleotide binding proteins and their lengths.

| Closure molecule | Manufacturer | Length | |
|---|---|---|---|
| TMAD | Sigma-Aldrich | ~2.05 | Å |
| (Tetramethylazodicarboxamide) | (D3648) | | |
| BMOE | Thermo Fisher | 8.0 | Å |
| (bismaleimidoethane) | Scientific | | |
| | (22323) | | |
| BMB | Thermo Fisher | 10.9 | Å |
| (1,4-bismaleimidobutane) | Scientific | | |
| | (22331) | | |
| BM(PEG)2 | Thermo Fisher | 14.7 | Å |
| (1,8-bismaleimido-diethyleneglycol) | Scientific | | |
| | (22336) | | |
| BM(PEG)3 | Thermo Fisher | 17.8 | Å |
| (1,11-bismaleimido-triethyleneglycol) | Scientific (22337) | | |

Immediately following this closure reaction, 12.5 μl of buffer (9.6 mM MgCl₂, 9.6 mM ATP, 3.2 M NaCl, 96 mM DTT), 1.8 μl of 100 μM RNA splint strand and 0.2 μl of nuclease free water was added and the solution was incubated for a further 25 minutes at room temperature. The resulting variant polynucleotide binding protein-bound sequencing adaptors were then purified using 3.7× volume of SPRI beads (Beckman Coulter), allowing them to bind the adaptors for 10-15 minutes, pelleting on a magnetic rack, then removing the supernatant and performing two washes with 20% PEG wash buffer. Finally, the purified polynucleotide binding protein sequencing adaptors were eluted in 50 μl of low salt elution buffer (50 mM Tris pH8, 10 mM NaCl) by incubating for 10 minutes at room temperature. These sequencing adaptors are now ready for step 2 of the library preparation.

### Library Preparation

The 1.3 kb RNA strand was prepared for sequencing with each of the variant sequencing adaptors and the control sequencing adaptor in two steps as outlined in SQK-RNA002 (Oxford Nanopore Technologies).

Briefly, a DNA oligo reverse transcription adaptor was ligated to the 1.3 kb RNA transcript using NEBNext Quick Ligation buffer (New England Biolabs) and T4 DNA ligase (New England Biolabs, M0202M). This product was then reverse transcribed using SuperScript III Reverse Transcriptase (Invitrogen), as per the manufacturer's instructions, and the resulting RNA: cDNA duplex was purified by incubating with Agencourt RNAClean XP beads (Beckman Coulter) and washing twice with 70% ethanol, as per SQK-RNA002 (Oxford Nanopore Technologies).

Following this, a second ligation reaction was performed using the NEBNext Quick Ligation buffer (New England Biolabs) and T4 DNA ligase (New England Biolabs, M0202M), whereby each variant sequencing adaptor and the control sequencing adaptor were independently ligated to the 1.3 kb RNA: cDNA molecule. The resulting products are the variant sequencing libraries and control sequencing library, which were purified and made ready for sequencing on a FLO-MIN106 MinION flowcell, as per SQK-RNA002 (Oxford Nanopore Technologies). A single MinION flowcell was run per library and at least two flowcells were run per library.

### Electrical Measurements

Electrical measurements were acquired on a FLO-MIN106 MinION flowcell and GridION sequencing device (Oxford Nanopore Technologies). A standard sequencing script was run and raw data collected in a bulk FAST5 file using MinKNOW software (Oxford Nanopore Technologies).

### Data Analysis

Following the sequencing experiment, the raw signal data from all variant and control sequencing libraries was inspected. The speed of individual strand translocation was calculated by dividing the length of the analyte over the duration of the read (measured in bases per second, bp/s). The median speed is the median bases per second of all strands within a condition as they translocated through the nanopore.

### Results

The results are shown in FIGS. 1-4. The results are also summarised in Tables 3 and 4 below.

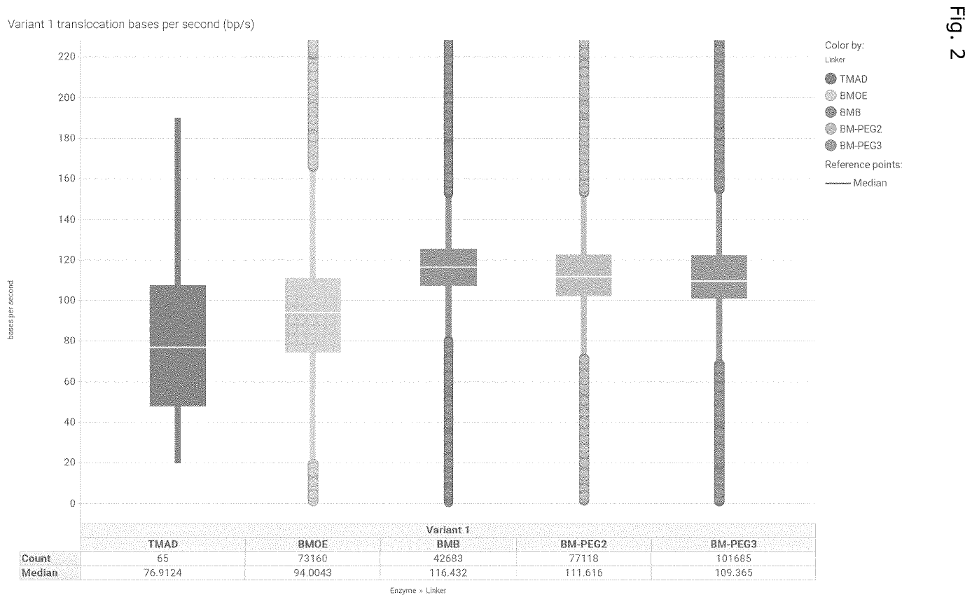

TABLE 3
Results in FIGS. 1 and 2

| | Linker | Median | Median base |
|---|---|---|---|
| Closure | Length (Å) | Duration (s) | pairs/second |
| TMAD | ~2.05 | 18.20 | 76.91 |
| BMOE | 8.00 | 14.89 | 94.00 |
| BMB | 10.90 | 12.02 | 116.43 |
| BM-PEG2 | 14.70 | 12.54 | 111.616 |
| BM-PEG3 | 17.80 | 12.80 | 109.365 |

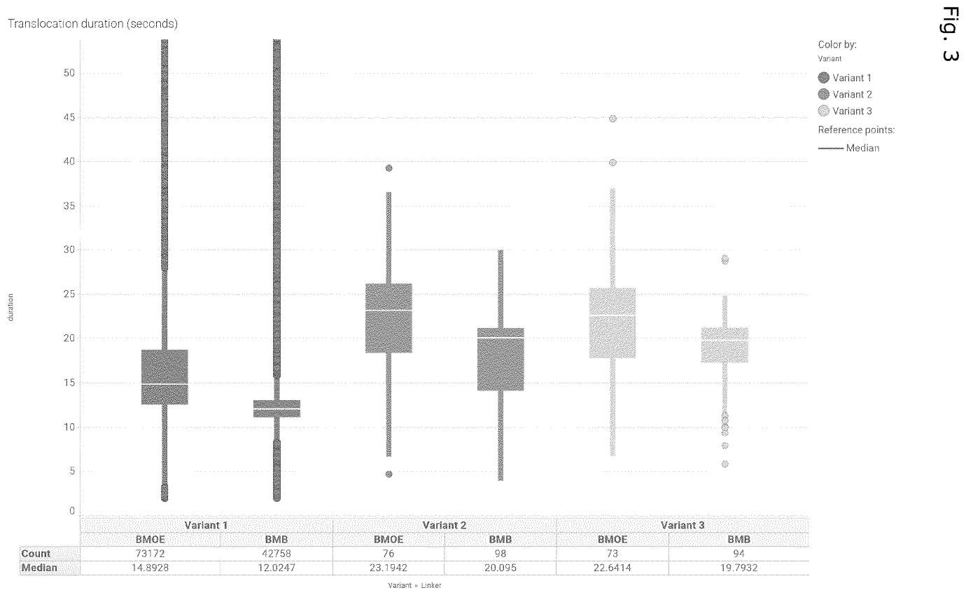

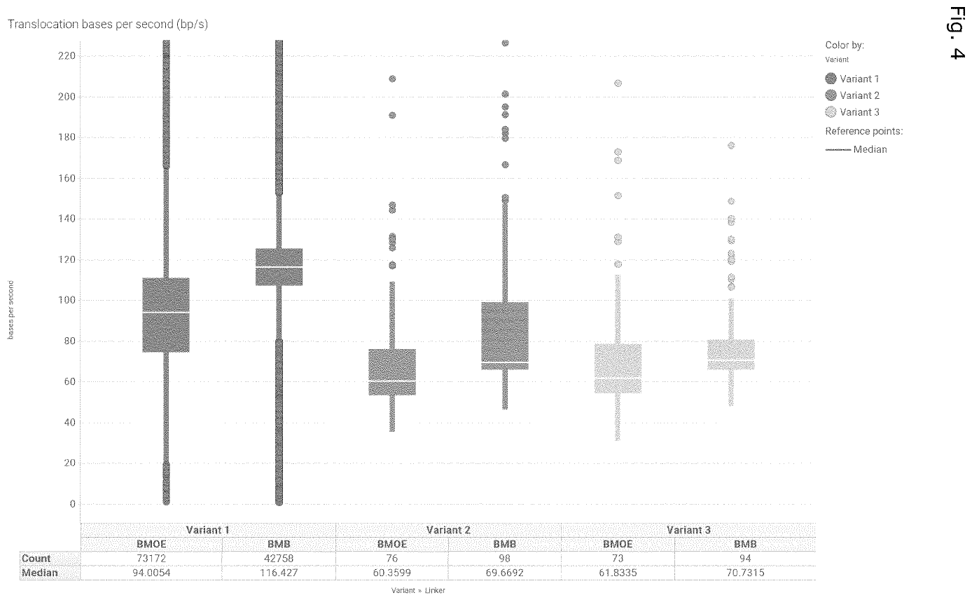

TABLE 4
Results in FIGS. 3 and 4

| | | Linker | Median | Median base |
|---|---|---|---|---|
| Enzyme | Closure | Length (Å) | Duration (s) | pairs/second |
| Variant 1 | BMOE | 8.00 | 14.89 | 94.00 |
| | BMB | 10.90 | 12.02 | 116.43 |
| Variant 2 | BMOE | 8.00 | 23.19 | 60.36 |
| | BMB | 10.90 | 20.09 | 69.67 |
| Variant 3 | BMOE | 8.00 | 22.64 | 61.83 |
| | BMB | 10.90 | 19.79 | 70.73 |

1. A modified NS3 helicase comprising a polynucleotide binding domain, wherein two or more parts of the helicase are connected via one or more attached linkers to form a covalently closed structure around the polynucleotide binding domain, wherein the one or more attached linkers are greater than about 8.0 angstroms (Å) in length and wherein the modified helicase retains its ability to control the movement of the polynucleotide.

2. A modified NS3 helicase according to claim 1, wherein the one or more attached linkers are at least about 10.0 Å in length.

3. A modified NS3 helicase according to claim 1 or 2, wherein the one or more attached linkers comprise 1,4-bis-maleimidobutane (BMB), bis-maleimide-PEG2 (BM-PEG2), BM-PEG3 or BM-PEG4.

4. A modified NS3 helicase according to any one of claims 1-3, wherein the NS3 helicase is derived from a dengue virus (NS3-DV), a yellow fever virus (NS3-YFV), or a hepatitis C virus (NS3-HCV).

5. A modified NS3 helicase according to claim 4, wherein the NS3 is derived from HCV JFH-1 (NS3-HCV-JFH1).

6. A modified NS3 helicase comprising a polynucleotide binding domain, wherein two or more parts of the helicase are connected via one or more attached linkers to form a covalently closed structure around the polynucleotide binding domain, wherein the NS3 helicase is derived from a dengue virus (NS3-DV), a yellow fever virus (NS3-YFV), or a hepatitis C virus (NS3-HCV), and wherein the modified helicase retains its ability to control the movement of the polynucleotide.

7. A modified NS3 helicase according to claim 6, wherein the NS3 is derived from HCV JFH-1 (NS3-HCV-JFH1).

8. A modified NS3 helicase according to claim 6 or 7, wherein the one or more attached linkers are as defined in any one of claims 1-3.

9. A method of increasing the speed at which a helicase is capable of controlling the movement of the polynucleotide, wherein the helicase comprises a polynucleotide binding domain, the method comprising modifying the helicase by connecting two or more parts of the helicase via one or more attached linkers greater than about 8.0 angstroms (Å) in length to form a covalently closed structure around the polynucleotide binding domain.

10. A method according to claim 9, wherein the one or more attached linkers are at least about 10.0 Å in length.

11. A method according to claim 9 or 10, wherein the helicase is a Dda, RecD, Upf1, PcrA, Rep, UvrD, Hel308, Mtr4, XPD, NS3, Mss116, Prp43, RecG, RecQ, T1R, RapA or Hef helicase.

12. A method according to any one of claims 9-11, wherein the helicase is a NS3 helicase.

13. A modified helicase which is capable of controlling the movement of a polynucleotide with an increased speed produced using a method according to any one of claims 9-12.

14. A construct comprising a modified helicase according to any one of claims 1-8 and 13 and an additional polynucleotide binding moiety, wherein the modified helicase is attached to the polynucleotide binding moiety and the construct has the ability to control the movement of a polynucleotide.

15. A construct according to claim 14, wherein the construct comprises two or more modified helicases according to any one of claims 1-8 and 13.

16. Use of a modified helicase according to any one of claims 1-8 and 13 or a construct according to claim 14 or 15 for controlling the movement of an analyte.

17. A method of controlling the movement of an analyte, comprising contacting the analyte with a modified helicase according to any one of claims 1-8 and 13 or a construct according to claim 14 or 15 and thereby controlling the movement of the analyte.

18. A method of determining the presence, absence or one or more characteristics of a target analyte, comprising:
(a) contacting the target analyte with a pore and a modified helicase according to any one of claims 1-8 and 13 or a construct according to claim 14 or 15 such that the modified helicase or construct controls the movement of the target analyte through the pore; and
(b) taking one or more measurements as the target analyte moves with respect to the pore wherein the measurements are indicative of one or more characteristics of the target analyte and thereby determining the presence, absence or one or more characteristics of the target analyte.

19. A method of forming a sensor for characterising a target analyte, comprising forming a complex between (a) a pore and (b) a modified helicase according to any one of claims 1-8 and 13 or a construct according to claim 14 or 15 and thereby forming a sensor for characterising the target analyte.

20. A sensor for characterising a target analyte, comprising a complex between (a) a pore and (b) a modified helicase according to any one of claims 1-8 and 13 or a construct according to claim 14 or 15.

21. A kit for characterising a target analyte comprising (a) a modified helicase according to any one of claims 1-8 and 13 or a construct according to claim 14 or 15 and (b) one or more loading moieties.

22. An apparatus for characterising target analytes in a sample, comprising (a) a plurality of pores and (b) a plurality of modified helicases according to any one of claims 1-8 and 13 or a plurality of constructs according to claim 14 or 15.

23. A method of producing a modified helicase according to any one of claims 1-8, comprising (a) providing a helicase and (b) modifying the helicase with one or more attached linkers to produce a modified helicase according to any one of claims 1-8.

24. A series of two or more helicases attached to a polynucleotide, wherein at least one of the two or more helicases is a modified helicase according to any one of claims 1-8 and 13.

25. A system comprising (a) a membrane comprising a pore, (c) a modified helicase according to any one of claims 1-8 and 13 or a construct according to claim 14 or 15, (b) means for applying a potential across the membrane and (c) means for detecting electrical or optical signals across the membrane.
